(19) United States
(12) Patent Application Publication
(10) Pub. No.: US 2017/0105969 A1
(43) Pub. Date: Apr. 20, 2017

(54) CENICRIVIROC FOR THE TREATMENT OF HIV-2 INFECTION

(71) Applicant: Tobira Therapeutics, Inc., South San Francisco, CA (US)

(72) Inventor: Eric LEFEBVRE, South San Francisco, CA (US)

(21) Appl. No.: 15/315,477

(22) PCT Filed: Jun. 2, 2015
(86) PCT No.: PCT/US15/33737

§ 371 Date: Dec. 1, 2016

Related U.S. Application Data
(60) Provisional application No. 62/006,492, filed on Jun. 2, 2014.

(51) Int. Cl. A61K 31/4178

(52) U.S. Cl. CPC A61K 31/4178

(57) ABSTRACT

Cenicriviroc (CVC) is an orally active antagonist of ligand binding to C—C chemokine receptor type 5 (CCR5) and C—C chemokine receptor type 2 (CCR2). CVC blocks the binding of RANTES, MIP-1α, and MIP-1β to CCR5, and of MCP-1/CCL2 to CCR2. Methods of treating HIV-2 infection and related conditions comprising administration of CVC are provided herein.

### CROSS-REFERENCE TO RELATED APPLICATIONS

This application claims the benefit under 35 U.S.C. §119(e) of U.S. Provisional Patent Application No. 62/006,492, filed Jun. 2, 2014. The foregoing application is incorporated herein by reference in its entirety.

### FIELD

The present disclosure relates to pharmaceutical compositions containing cenicriviroc, methods for the preparation thereof, and their use in the treatment of HIV-2 infection.

### BACKGROUND

Cenicriviroc (also known as CVC) has the common name of (S,E)-8-(4-(2-Butoxyethoxy)phenyl)-1-(2-methylpropyl)-N-(4-(((1-propyl-1H-imidazol-5-yl)methyl)sulfinyl)phenyl)-1,2,3,4-tetrahydrobenzo[b]azocine-5-carboxamide or 8-[4-(2-butoxyethoxy)phenyl]-1,2,3,4-tetrahydro-1-(2-methylpropyl)-N-[4-[(S)-[(1-propyl-1H-imidazol-5-yl)methyl]sulfinyl]phenyl]-1-Benzazocine-5-carboxamide. The chemical structure of cenicriviroc mesylate appears in FIG. 1. Cenicriviroc binds to and inhibits the activity of the C—C chemokine receptor type 2 (CCR2) and C—C chemokine receptor type 5 (CCR5) receptors (24). The CCR5 receptor plays a role in entry of viruses such as Human Immunodeficiency Virus (HIV) into the cell.

Over the past 2 decades, HIV (human immunodeficiency virus) antiretrovirals (ARVs) have been developed for treatment of HIV and AIDS (acquired immune deficiency syndrome). Combined use of ARVs for the treatment of HIV-1 and AIDS is recommended by all HIV treatment guidelines. However, treatment options for HIV-2 are still limited. HIV-2 is naturally resistant to non-nucleoside reverse transcriptase inhibitors, fusion inhibitor, and to some protease inhibitors. Development of a new ARVs with activity on HIV-2 is needed.

CD4 is a well established receptor utilized by HIV to invade target cells such as lymphocytes. In addition to CD4, other co-receptors are also involved in HIV entry: CCR5 and CXCR4 are G-coupled protein conjugated chemokine receptors, have been shown to play a critical role for infection, transmission and/or progression of HIV disease. It has been reported that a man having resistance to infection even after repeated exposures to the virus had a mutation in which CCR5 gene was deleted homologically. Individuals with the CCR5-Δ32 mutation, which results in a truncated protein that is not expressed on the cell surface, resist infection by HIV-1 and have no obvious health problems. These observations suggest that a CCR5 antagonist may be an effective anti-HIV-1 drug.

Thus, CCR5 antagonists have a potential to provide a new HIV medicine, and examples of synthesis of new anilide derivatives having CCR5 antagonist activity have been reported in, for example, PCT/JP98/05708 (WO99/32100), Japanese Patent Application No. 10-234388 (WO00/10965), and Japanese Patent Application No. 10-363404 (PCT/JP99/07148). On Aug. 6, 2007, the Food and Drug Administration (FDA) approved maraviroc (SELZENTRY), the first CCR5 co-receptor antagonist in this new class, to be used in combination with other antiretroviral products for the treatment of adults infected with CCR5-tropic HIV-1. Further, a compound having CCR5 antagonist activity has been described as useful as a preventative medicine of HIV/AIDS in JP 2001-026586 A, but said compound has a different structure from the compound of the present invention.

### SUMMARY OF THE INVENTION

The present invention provides a class of bicyclic compound including CVC to treat and/or prevent HIV-2 infection in a patient in need thereof.

In one embodiment, the invention provides a method of treating HIV-2 infection in a patient in need of such treatment, comprising administering to the patient a therapeutically effective amount of a compound of formula (I):

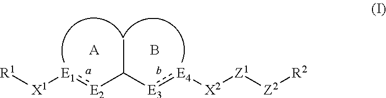

wherein
R¹ is a cyclic 5- to 6-membered ring which may be substituted;
X¹ is a bond;
rings A and B, together with the variables a, b, E₁, E₂, E₃, and E₄, form a benzoazocine ring system;
X² is a bivalent chain group whose straight chain moiety is constituted of 1 to 4 atoms;
Z¹ is a bond or a bivalent cyclic group;
Z² is a bond or a bivalent group; and
R² is
   (1) an amino group which may be substituted and whose nitrogen atoms may be converted to quaternary ammonium or oxide,
   (2) a nitrogen-containing heterocyclic group which may be substituted, may contain a sulfur or oxygen atom as a ring constituent atom, and whose nitrogen atom may be converted to quaternary ammonium or oxide,
   (3) a group of the formula:

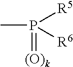

wherein k is 0 or 1; when k is 0, the phosphorus atom may form a phosphonium salt; each of R⁵ and R⁶ is a hydrocarbon group which may be substituted, a hydroxy group or an amino group which may be substituted; and R⁵ and R⁶ may form a ring with the adjacent phosphorus atom,
(4) an amidino group which may be substituted, or
(5) a guanidino group which may be substituted;
   or a salt thereof, to a mammal in need thereof.

In one embodiment, the present invention provides a method of inhibiting HIV-2 viral replication in a patient in need thereof, comprising administering to the patient a therapeutically effective amount of a compound of formula (I):

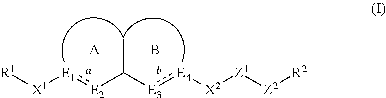

wherein
R¹ is a cyclic 5- to 6-membered ring which may be substituted;
X¹ is a bond;
rings A and B, together with the variables a, b, E₁, E₂, E₃, and E₄, form a benzoazocine ring system;
X² is a bivalent chain group whose straight chain moiety is constituted of 1 to 4 atoms;
Z¹ is a bond or a bivalent cyclic group;
Z² is a bond or a bivalent group; and
R² is
   (1) an amino group which may be substituted and whose nitrogen atoms may be converted to quaternary ammonium or oxide,
   (2) a nitrogen-containing heterocyclic group which may be substituted, may contain a sulfur or oxygen atom as a ring constituent atom, and whose nitrogen atom may be converted to quaternary ammonium or oxide,
   (3) a group of the formula:

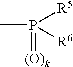

wherein k is 0 or 1; when k is 0, the phosphorus atom may form a phosphonium salt; each of R⁵ and R⁶ is a hydrocarbon group which may be substituted, a hydroxy group or an amino group which may be substituted; and R⁵ and R⁶ may form a ring with the adjacent phosphorus atom,
(4) an amidino group which may be substituted, or
(5) a guanidino group which may be substituted;
   or a salt thereof, to a mammal in need thereof.

In one embodiment, the present invention provides a method of inhibiting HIV-2 binding to a target cell in a patient in need thereof, comprising administering to the patient a therapeutically effective amount of a compound of formula (I):

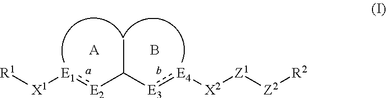

wherein
R¹ is a cyclic 5- to 6-membered ring which may be substituted;
X¹ is a bond;
rings A and B, together with the variables a, b, E₁, E₂, E₃, and E₄, form a benzoazocine ring system;
X² is a bivalent chain group whose straight chain moiety is constituted of 1 to 4 atoms;
Z¹ is a bond or a bivalent cyclic group;
Z² is a bond or a bivalent group; and
R² is
   (1) an amino group which may be substituted and whose nitrogen atoms may be converted to quaternary ammonium or oxide,
   (2) a nitrogen-containing heterocyclic group which may be substituted, may contain a sulfur or oxygen atom as a ring constituent atom, and whose nitrogen atom may be converted to quaternary ammonium or oxide,
   (3) a group of the formula:

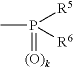

wherein k is 0 or 1; when k is 0, the phosphorus atom may form a phosphonium salt; each of R⁵ and R⁶ is a hydrocarbon group which may be substituted, a hydroxy group or an amino group which may be substituted; and R⁵ and R⁶ may form a ring with the adjacent phosphorus atom,
(4) an amidino group which may be substituted, or
(5) a guanidino group which may be substituted;
   or a salt thereof, to a mammal in need thereof.

In another embodiment, the present invention provides a method for treating a HIV-2 infection comprising administering an effective amount of (S)-(8)-[4-(2-Butoxyethoxy)phenyl]-1-isobutyl-N-(4-{[(1-propyl-1H-imidazol-5-yl)methyl]sulfinyl}phenyl)-1,2,3,4-tetrahydro-1-benzazocine-5-carboxamide or a salt thereof to a subject in need thereof. In a further embodiment, the present invention provides a method for treating a HIV-2 infection comprising administering an effective amount of a mesylate salt of the compound of the invention to a subject in need thereof.

In another embodiment, the present invention provides a method of inhibiting HIV-2 viral replication comprising administering an effective amount of (S)-(8)-[4-(2-Butoxyethoxy)phenyl]-1-isobutyl-N-(4-{[(1-propyl-1H-imidazol-5-yl)methyl]sulfinyl}phenyl)-1,2,3,4-tetrahydro-1-benzazocine-5-carboxamide or a salt thereof to a subject in need thereof. In a further embodiment, the present invention provides a method of inhibiting HIV-2 viral replication comprising administering an effective amount of a mesylate salt of the compound of the invention to a subject in need thereof.

In a further embodiment, the present invention provides a method of inhibiting HIV-2 binding to a target cell comprising administering an effective amount of (S)-(8)-[4-(2-Butoxyethoxy)phenyl]-1-isobutyl-N-(4-{[(1-propyl-1H-imidazol-5-yl)methyl]sulfinyl}phenyl)-1,2,3,4-tetrahydro-1-benzazocine-5-carboxamide or a salt to a subject in need thereof. In yet a further embodiment, the present invention provides a method of inhibiting HIV-2 binding to a target cell comprising administering an effective amount of a mesylate salt of the compound of the invention to a subject in need thereof. In yet a further embodiment, the binding of HIV-2 to a cell-surface receptor is blocked and/or inhibited. In another further embodiment, the cell-surface receptor is CCR5. In another further embodiment, the cell-surface receptor is CCR2.

In a further embodiment, the compounds or compositions of the invention are used in combination with one or more agents that purge latent HIV reservoirs. For example, the compounds or compositions of the invention can be co-administered orally or added to blood for transfusion or to blood derivatives with one or more agents that purge latent HIV reservoirs. In yet another embodiment, the compounds or compositions of the invention are administered at the same time of or within 1 hour after transfusion or use of blood derivatives. The one or more agents that purge latent HIV reservoirs can be either proteins (e.g., Interleukin 7) or compounds (e.g., prostratin) which can stimulate inactive cells infected with HIV to produce new virus particles that are susceptible to antiretroviral therapy.

### BRIEF DESCRIPTION OF THE DRAWINGS

FIG. 1 is the chemical formula of cenicriviroc mesylate.

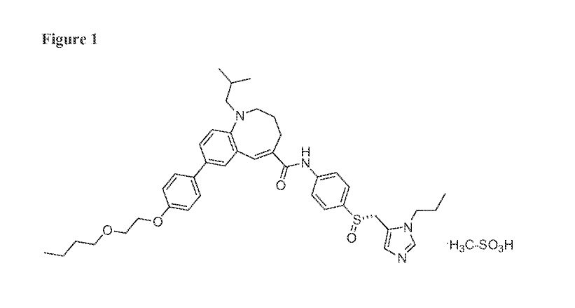

FIG. 2 is a graph comparing the absolute bioavailability, in beagle dogs, of cenicriviroc mesylate compounded as an oral solution with that of cenicriviroc mesylate prepared by wet granulation and mixed with various acid solubilizer excipients.

FIG. 3 is a graph of the total impurity and degradant content of different cenicriviroc formulations subjected to accelerated stability testing at 40° C. and 75% relative humidity when packaged with a desiccant.

FIG. 4 is a dynamic vapor sorption isotherm for different cenicriviroc formulations.

FIG. 5 shows the absorption of cenicriviroc from different formulations at three pre-treatment states in beagle dogs.

FIG. 6 shows the beagle dog absolute bioavailability of cenicriviroc (CVC) and lamivudine (3TC) in combination tablets.

FIG. 7 shows the median Changes in HIV-1 RNA levels from baseline by cohort and study day—Study 201.

FIG. 8 shows the proportion of subjects with HIV-1 RNA<50 Copies/mL over time up to Week 48—Snapshot Algorithm—ITT—Study 202.

FIG. 9 shows the inhibition of HIV-2 viral replication after exposure to maraviroc. For the 13 R5 clinical isolates, the median EC₅₀ is 0.80 nM, with the interquartiles of 0.48 to 1.39 nM; the median MPI is 93%, with the interquartiles of 84-98%. For the two mixed R5/X4 clinical isolates, the median EC₅₀ is 9.40 nM and greater 1000 nM, and the median MPI is 55% and 12%. For the X4 clinical isolate, the median EC₅₀ is greater than 1000 nM, and the median MPI is 0%.

FIGS. 10 A and B show the percent viral inhibition for HIV-2 (Panel A) and HIV-1 (Panel B).

FIG. 11 shows dose response curves for cenicriviroc-dependent inhibition of HIV-2 primary clinical isolates.

### DETAILED DESCRIPTION

It should be understood that singular forms such as “a,” “an,” and “the” are used throughout this application for convenience, however, except where context or an explicit statement indicates otherwise, the singular forms are intended to include the plural. Further, it should be understood that every journal article, patent, patent application, publication, and the like that is mentioned herein is hereby incorporated by reference in its entirety and for all purposes. All numerical ranges should be understood to include each and every numerical point within the numerical range, and should be interpreted as reciting each and every numerical point individually. The endpoints of all ranges directed to the same component or property are inclusive, and intended to be independently combinable.

### DEFINITIONS

Except for the terms discussed below, all of the terms used in this Application are intended to have the meanings that one of skill in the art at the time of the invention would ascribe to them.

“About” includes all values having substantially the same effect, or providing substantially the same result, as the reference value. Thus, the range encompassed by the term “about” will vary depending on context in which the term is used, for instance the parameter that the reference value is associated with. Thus, depending on context, “about” can mean, for example, ±10%, ±5%, ±4%, ±3%, ±2%, ±1%, or ±less than 1%. Importantly, all recitations of a reference value preceded by the term “about” are intended to also be a recitation of the reference value alone. Notwithstanding the preceding, in this application the term “about” has a special meaning with regard to pharmacokinetic parameters, such as area under the curve (including AUC, AUCₜ, and AUC_{∞}) Cₘₐₓ, Tₘₐₓ, and the like. When used in relationship to a value for a pharmacokinetic parameter, the term “about” means from 80% to 125% of the reference parameter.

“Cenicriviroc” refers to the chemical compound (S)-8-[4-(2-Butoxyethoxy)phenyl]-1-isobutyl-N-(4-{[(1-propyl-1H-imidazol-5-yl)methyl]sulfinyl}phenyl)-1,2,3,4-tetrahydro-1-benzazocine-5-carboxamide (structure shown below). Cenicriviroc also has a CAS registry number of 497223-25-3. Details of the composition of matter of cenicriviroc are disclosed in US Patent Application Publication No. 2012/0232028 which is hereby incorporated by reference in its entirety for all purposes. Details of related formulations are disclosed in International Application No. PCT/US2014/038211, filed May 15, 2014, which is hereby incorporated by reference in its entirety for all purposes.

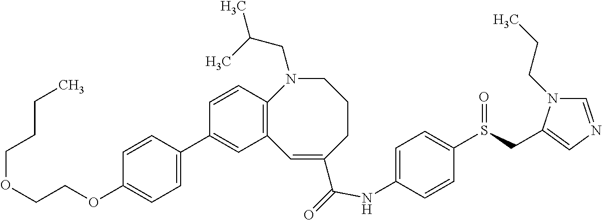 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1]

“Compound of the present invention” or “the present compound” refers to cenicriviroc (CVC) or a salt or solvate thereof.

“Substantially similar” means a composition or formulation that resembles the reference composition or formulation to a great degree in both the identities and amounts of the composition or formulation.

“Pharmaceutically acceptable” refers to a material or method that can be used in medicine or pharmacy, including for veterinary purposes, for example, in administration to a subject.

“Salt” and “pharmaceutically acceptable salt” includes both acid and base addition salts. “Acid addition salt” refers to those salts that retain the biological effectiveness and properties of the free bases, which are not biologically or otherwise undesirable, and which are formed with inorganic acids and organic acids. “Base addition salt” refers to those salts that retain the biological effectiveness and properties of the free acids, which are not biologically or otherwise undesirable, and which are prepared from addition of an inorganic base or an organic base to the free acid. Examples of pharmaceutically acceptable salts include, but are not limited to, mineral or organic acid addition salts of basic residues such as amines; alkali or organic addition salts of acidic residues; and the like, or a combination comprising one or more of the foregoing salts. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, or a combination comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; or a combination comprising one or more of the foregoing salts.

In one embodiment, the acid addition salt of cenicriviroc is cenicriviroc mesylate, e.g., (S)-8-[4-(2-Butoxyethoxy)phenyl]-1-isobutyl-N-(4-{[(1-propyl-1H-imidazol-5-yl)methyl]sulfinyl}phenyl)-1,2,3,4-tetrahydro-1-benzazocine-5-carboxamide monomethanesulfonoate. In one embodiment, the cenicriviroc mesylate is a crystalline material, such as a pale greenish-yellow crystalline powder. In one embodiment, the cenicriviroc mesylate is freely soluble in glacial acetic acid, methanol, benzyl alcohol, dimethylsulfoxide, and N,N-dimethylformamide; soluble in pyridine and acetic anhydride; and sparingly soluble in 99.5% ethanol; slightly soluble in acetonitrile, 1-octanol, and tetrahydrofuran; and practically insoluble in ethyl acetate and diethylether. In one embodiment, the cenicriviroc mesylate is freely soluble in aqueous solution from pH 1 to 2; sparingly soluble at pH 3 and practically insoluble from pH 4 to 13 and in water.

“Solvate” means a complex formed by solvation (the combination of solvent molecules with molecules or ions of the active agent of the present invention), or an aggregate that consists of a solute ion or molecule (the active agent of the present invention) with one or more solvent molecules. In the present invention, the preferred solvate is hydrate.

“Pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents or excipients therefor.

“Treating” includes ameliorating, mitigating, and reducing the instances of a disease or condition, or the symptoms of a disease or condition.

“Administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, and transdermal. “Administering” can also include prescribing or filling a prescription for a dosage form comprising a particular compound. “Administering” can also include providing directions to carry out a method involving a particular compound or a dosage form comprising the compound.

“Therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

As used herein, “CCR5” or “R5”, is a chemokine receptor which binds members of the C—C group of chemokines and whose amino acid sequence comprises that provided in Genbank Accession Number 1705896 and related polymorphic variants. As used herein, CCR5 includes, without limitation, extracellular portions of CCR5 capable of binding the HIV envelope protein. “CCR5” and “CCR5 receptor” are used synonymously.

“CXCR4” or “X4” is a chemokine receptor which binds members of the C—X—C group of chemokines and whose amino acid sequence comprises that provided in Genbank Accession No 400654 and related polymorphic variants. As used herein, CXCR4 includes extracellular portions of CXCR4 capable of binding the HIV envelope protein.

“HIV” refers to the human immunodeficiency virus. The human immunodeficiency virus (HIV) may be either of the two known types of HIV (HIV-1 or HIV-2). The virus may represent any of the known major subtypes (classes A, B, C, D, E, F, G, H, or J), outlying subtype (Group O), or an as yet to be determined subtype of HIV.

### HIV-2

HIV-2 is a retrovirus that infects between one and three million people worldwide and has a mortality rate estimated to be a third lower than that for HIV-1 (Whittle et al. (1994)). The HIV-1 and HIV-2 genomes differ by about 50-60% at the nucleotide levels. The HIV viruses, and the closely related SIV viruses, possess a lipid membrane that fuses with the cell membrane to allow the virus core and RNA genome access to the cell cytoplasm. Glycoprotein spikes on the surface of virus particles attach to specific receptors at the cell surface and induce fusion of viral and cellular membranes. HIV-1, HIV-2 and SIV strains can infect cells by interacting with the cell surface CD4 receptor and seven-transmembrane coreceptors to infect cells, and there are marked differences between the coreceptor used by the viruses. HIV-2 can exploit a broad range of coreceptors for infection of CD4⁺ cell lines, including CCR5 and CXCR4 as well as alternate coreceptors CCR1, CCR2b, CCR5, CPR15 (BOB), CCR5, and CXCR6 (BONZO). Further, many HIV-2 strains can also infect CD4⁻ cells expressing either CCR5 or CXCR4. (Reeves et al. (1999)).

HIV-2 is naturally resistant to non-nucleoside reverse transcriptase inhibitors, a fusion inhibitor, and to some protease inhibitors. (Visseaux et al. (2012) and therefore treatment options for HIV-2 are limited relative to those for HW-1. One proposed method of treating and/or preventing HW-2 infection is the development and use of molecules that inhibit entry of HIV-2 into a target cell. Having a class of molecules with a cellular, not a viral, target is beneficial since the cell target is not impacted by HW genetic diversity. This may provide a new therapeutic opportunity for treatment of HIV-2 infection.

Provided herein are compositions and methods for treating HW-2 infection. The present invention provides a novel CCR antagonist that inhibits entry of HIV-2 in target cells. The compounds of the present invention have excellent CCR antagonistic action, in particular, CCR5 and/or CCR2 antagonistic action, especially, a strong CCR5 antagonistic action, and may be used, for example, for prevention and treatment of a variety of human HIV infectious diseases, for example, HIV/AIDS. The compounds of the present invention have low toxicity and can be used safely. A pharmaceutical composition containing the compounds of the present invention can be used, for example, as a CCR5 antagonist, as a preventive and therapeutic medicine for HW infection and for avoiding disease progression to AIDS. In a further embodiment, a pharmaceutical composition containing the compounds of the present invention can be used, for example, as a CCR5 antagonist, as a preventive and therapeutic medicine for HIV infection and for suppression on disease progression of HW infection. In a particular embodiment, the HIV infection is HIV-2.

Certain embodiments include methods for monitoring and/or predicting the treatment efficacy of the present treatment as described herein. Such methods include detecting the level of one or more biological molecules in a subject (or in a biological sample from the subject) treated for HIV infection, wherein an increase or decrease in the level of one or more biological molecules compared to a predetermined standard level indicates or is predictive of the treatment efficacy of the present treatment.

### Dosages and Administration:

A dosage of a particular subject can be determined according to the subject's age, weight, general health conditions, sex, meal, administration time, administration route, excretion rate and the degree of particular disease conditions to be treated by taking into consideration of these and other factors.

The present invention provides a method of treatment, wherein the cenicriviroc or a salt or solvate thereof is formulated as an oral composition.

The present invention provides a method of treatment, wherein the cenicriviroc or a salt or solvate thereof is administered once per day or twice per day, or more. The dosage form can be administered for a duration of time sufficient to prevent or treat HIV disease or avoid progression to AIDS.

In the case of oral administration, a daily dosage is in a range of about 5 to 1000 mg as the active ingredient (i.e. as the compound of the invention) per an adult of body weight of 50 kg. In one embodiment, the daily dosage is in a range of about 10 to 600 mg. In another embodiment, the daily dosage is in a range of about 20 to 400 mg. In another embodiment, the daily dosage is in a range of about 30 to 250 mg. The medicine may be administered once or in 2 to 3 divided doses a day.

The cenicriviroc or a salt or solvate thereof may be formulated into any dosage form suitable for oral or injectable administration. When the compound is administered orally, it can be formulated into solid dosage forms for oral administration, for example, tablets, capsules, pills, granules, and so on. It also can be formulated into liquid dosage forms for oral administration, such as oral solutions, oral suspensions, syrups and the like. The term “tablets” as used herein, refers to those solid preparations which are prepared by homogeneously mixing and pressing the compounds and suitable auxiliary materials into circular or irregular troches, mainly in common tablets for oral administration, including also buccal tablets, sublingual tablets, buccal wafer, chewable tablets, dispersible tablets, soluble tablets, effervescent tablets, sustained-release tablets, controlled-release tablets, enteric-coated tablets and the like. The term “capsules” as used herein, refers to those solid preparations which are prepared by filling the compounds, or the compounds together with suitable auxiliary materials into hollow capsules or sealing into soft capsule materials. According to the solubility and release property, capsules can be divided into hard capsules (regular capsules), soft capsules (soft shell capsules), sustained-release capsules, controlled-release capsules, enteric-coated capsules and the like. The term “pills” as used herein, refers to spherical or near-spherical solid preparations which are prepared by mixing the compounds and suitable auxiliary materials via suitable methods, including dropping pills, dragee, pilule and the like. The term “granules” as used herein, refers to dry granular preparations which are prepared by mixing the compounds and suitable auxiliary materials and have a certain particle size. Granules can be divided into soluble granules (generally referred to as granules), suspension granules, effervescent granules, enteric-coated granules, sustained-release granules, controlled-release granules and the like. The term “oral solutions” as used herein, refers to a settled liquid preparation which is prepared by dissolving the compounds in suitable solvents for oral administration. The term “oral suspensions” as used herein, refers to suspensions for oral administration, which are prepared by dispersing the insoluble compounds in liquid vehicles, also including dry suspension or concentrated suspension. The term “syrups” as used herein, refers to a concentrated sucrose aqueous solution containing the compounds. The injectable dosage form can be produced by the conventional methods in the art of formulations, and aqueous solvents or non-aqueous solvents may be selected. The most commonly used aqueous solvent is water for injection, as well as 0.9% sodium chloride solution or other suitable aqueous solutions. The commonly used non-aqueous solvent is vegetable oil, mainly soy bean oil for injection, and others aqueous solutions of alcohol, propylene glycol, polyethylene glycol, and etc.

In one embodiment, a pharmaceutical composition comprising cenicriviroc or a salt thereof and fumaric acid is provided. In certain embodiments, the cenicriviroc or salt thereof is cenicriviroc mesylate.

In further embodiments, the weight ratio of cenicriviroc or salt thereof to fumaric acid is from about 7:10 to about 10:7, such as from about 8:10 to about 10:8, from about 9:10 to about 10:9, or from about 95:100 to about 100:95. In other further embodiments, the fumaric acid is present in an amount of from about 15% to about 40%, such as from about 20% to about 30%, or about 25%, by weight of the composition. In other further embodiments, the cenicriviroc or salt thereof is present in an amount of from about 15% to about 40%, such as from about 20% to about 30%, or about 25%, by weight of the composition.

In other further embodiments, the composition of cenicriviroc or a salt thereof and fumaric acid further comprises one or more fillers. In more specific embodiments, the one or more fillers are selected from microcrystalline cellulose, calcium phosphate dibasic, cellulose, lactose, sucrose, mannitol, sorbitol, starch, and calcium carbonate. For example, in certain embodiments, the one or more fillers is microcrystalline cellulose. In particular embodiments, the weight ratio of the one or more fillers to the cenicriviroc or salt thereof is from about 25:10 to about 10:8, such as from about 20:10 to about 10:10, or about 15:10. In other particular embodiments, the one or more fillers are present in an amount of from about 25% to about 55%, such as from about 30% to about 50% or about 40%, by weight of the composition. In other further embodiments, the composition further comprises one or more disintegrants. In more specific embodiments, the one or more disintegrants are selected from cross-linked polyvinylpyrrolidone, cross-linked sodium carboxymethyl cellulose, and sodium starch glycolate. For example, in certain embodiments, the one or more disintegrants is cross-linked sodium carboxymethyl cellulose. In particular embodiments, the weight ratio of the one or more disintegrants to the cenicriviroc or salt thereof is from about 10:10 to about 30:100, such as about 25:100. In other particular embodiments, the one or more disintegrants are present in an amount of from about 2% to about 10%, such as from about 4% to about 8%, or about 6%, by weight of the composition. In other further embodiments, the composition further comprises one or more lubricants. In more specific embodiments, the one or more lubricants are selected from talc, silica, stearin, magnesium stearate, and stearic acid. For example, in certain embodiments, the one or more lubricants is magnesium stearate. In particular embodiments, the one or more lubricants are present in an amount of from about 0.25% to about 5%, such as from about 0.75% to about 3%, or about 1.25%, by weight of the composition.

In other further embodiments, the composition of cenicriviroc or a salt thereof and fumaric acid is substantially similar to that of Table 1. In other further embodiments, the composition of cenicriviroc or a salt thereof and fumaric acid is substantially similar to that of Tables 2 and 3. In other further embodiments, any of the compositions of cenicriviroc or a salt thereof and fumaric acid is produced by a process involving dry granulation. In other further embodiments, any of the compositions of cenicriviroc or a salt thereof and fumaric acid has a water content of no more than about 4% by weight, such as no more than 2% by weight, after six weeks exposure to about 40° C. at about 75% relative humidity when packaged with desiccant. In other further embodiments, any of the above-mentioned compositions has a total impurity level of no more than about 2.5%, such as no more than 1.5%, after 12 weeks of exposure to 40° C. at 75% relative humidity when packaged with desiccant. In other further embodiments, the cenicriviroc or salt thereof of any of the above-mentioned compositions has a mean absolute bioavailability after oral administration that is substantially similar to the bioavailability of the cenicriviroc or salt thereof in a solution after oral administration. In yet further embodiments, the cenicriviroc or salt thereof has an absolute bioavailability of about 10% to about 50%, such as about 27%, in beagle dogs.

In another embodiment, a pharmaceutical formulation is provided that comprises a composition of cenicriviroc or a salt thereof and fumaric acid. In further embodiments, the composition in the formulation can be in the form of a granulate. In other further embodiments, the composition in the formulation is disposed in a capsule shell. In other further embodiments, the composition of the formulation is disposed in a sachet. In other further embodiments, the composition of the formulation is a tablet or a component of a tablet. In still other further embodiments, the composition of the formulation is one or more layers of a multi-layered tablet. In other further embodiments, the formulation comprises one or more additional pharmaceutically inactive ingredients. In other further embodiments, the formulation is substantially similar to that of Table 8. In other further embodiments, a tablet having a composition substantially similar to of Table 8 is provided. In other further embodiments, any of the above embodiments are coated substrates. In another embodiment, methods for preparing any of the above-mentioned embodiments are provided. In further embodiments, the method comprises admixing cenicriviroc or a salt thereof and fumaric acid to form an admixture, and dry granulating the admixture. In other further embodiments, the method further comprises admixing one or more fillers with the cenicriviroc or salt thereof and fumaric acid to form the admixture. In other further embodiments, the method further comprises admixing one or more disintegrants with the cenicriviroc or salt thereof and fumaric acid to form the admixture. In other further embodiments, the method further comprises admixing one or more lubricants with the cenicriviroc or salt thereof and fumaric acid to form the admixture. In other further embodiments, the method further comprises compressing the dry granulated admixture into a tablet. In other further embodiments, the method comprises filling a capsule with the dry granulated admixture.

The compounds or compositions of the invention can be used in combination with one or more agents that purge latent HIV reservoirs. In one embodiment, the compounds or compositions of the invention can be co-administered orally with one or more agents that purge latent HIV reservoirs. In one embodiment, the compound of the invention can be included or used in combination with one or more agents that purge latent HIV reservoirs and added to blood for transfusion or blood derivatives. Usually, blood for transfusion or blood derivatives are produced by mixing blood obtained form plural persons and, in some cases, uninfected cells are contaminated with cells infected with HIV virus. In such a case, uninfected cells are likely to be infected with HIV virus. When the compound of the present invention is added to blood for transfusion or blood derivatives along with one or more agents that purge latent HIV reservoirs, infection and proliferation of the virus can be prevented or controlled. Especially, when blood derivatives are stored, infection and proliferation of the virus is effectively prevented or controlled by addition of the compound of the present invention. In addition, when blood for transfusion or blood derivatives contaminated with HIV virus are administered to a person, infection and proliferation of the virus in the person's body can be prevented by adding the compound of the invention to the blood or blood derivatives in combination with one or more agents that purge latent HIV reservoirs. For example, usually, for preventing HIV infectious disease upon using blood or blood derivatives by oral administration, a dosage is in a range of about 0.02 to 50 mg/kg, preferably about 0.05 to 30 mg/kg, and more preferably about 0.1 to 10 mg/kg as the CCR antagonist per an adult of body weight of about 60 kg, and the medicine may be administered once or 2 to 3 doses a day. As a matter of course, although the dosage range can be controlled on the basis of unit dosages necessary for dividing the daily dosage, as described above, a dosage of a particular subject can be determined according to the subject's age, weight, general health conditions, sex, meal, administration time, administration route, excretion rate and the degree of particular disease conditions to be treated by taking into consideration of these and other factors. In this case, the administration route is also appropriately selected and, the medicine for preventing HIV infectious disease of the present invention may be added directly to blood for transfusion or blood derivatives before transfusion or using blood derivatives. In such a case, desirably, the medicine of the present invention is mixed with blood or blood derivatives immediately to 24 hours before, preferably immediately to 12 hours before, more preferably immediately to 6 hours before transfusion or using blood derivatives.

Aside from blood for transfusion or blood derivatives, when the compositions of the invention is administered together with the blood for transfusion or blood derivatives and/or other active agents, the medicine is administered preferably at the same time of, to 1 hour before transfusion or using the blood derivatives. More preferably, the medicine is administered once to 3 times per day and the administration is continued 4 weeks.

### Combination Therapy:

The compound of the invention may be used alone or in combination with one or more additional active agents. The one or more additional active agents may be any compound, molecule, or substance which can exert therapeutic effect to a subject in need thereof. The one or more additional active agents may be “co-administered”, i.e, administered together in a coordinated fashion to a subject, either as separate pharmaceutical compositions or admixed in a single pharmaceutical composition. By “co-administered”, the one or more additional active agents may also be administered simultaneously with the present compound, or be administered separately with the present compound, including at different times and with different frequencies. The one or more additional active agents may be administered by any known route, such as orally, intravenously, intramuscularly, nasally, and the like; and the therapeutic agent may also be administered by any conventional route. In many embodiments, at least one and optionally both of the one or more additional active agents may be administered orally.

These one or more additional active agents include, but are not limited to, one or more anti-fibrotic agents, antiretroviral agents, immune system suppressing agents, and any combinations thereof. When two or more medicines are used in combination, dosage of each medicine is commonly identical to the dosage of the medicine when used independently, but when a medicine interferes with metabolism of other medicines, the dosage of each medicine is properly adjusted. Each medicine may be administered simultaneously or separately in a time interval of less than 12 hours. A dosage form as described herein, such as a capsule, can be administered at appropriate intervals. For example, once per day, twice per day, three times per day, and the like. In particular, the dosage form is administered once or twice per day. Even more particularly, the dosage form is administered once per day. Also, more particularly, the dosage form is administered twice per day.

In one embodiment, the one or more antiretroviral agents include, but are not limited to, entry inhibitors, nucleoside reverse transcriptase inhibitors, nucleotide reverse transcriptase inhibitors, non-nucleoside reverse transcriptase inhibitors, protease inhibitors, integrase inhibitors, maturation inhibitors (e.g., GSK2838232), and combinations thereof. In one embodiment, the one or more additional antiretroviral agents include, but are not limited to, lamivudine, efavirenz, raltegravir, vivecon, bevirimat, alpha interferon, zidovudine, abacavir, lopinavir, ritonavir, tenofovir, tenofovir disoproxil or its fumarate salt, tenofovir alafenamide or its fumarate salt, emtricitabine, elvitegravir, cobicistat, darunavir, atazanavir, rilpivirine, dolutegravir, and a combination thereof.

In one embodiment, the one or more immune system suppressing agents include, but are not limited to, cyclosporine, tacrolimus, prednisolone, hydrocortisone, sirolimus, everolimus, azathioprine, mycophenolic acid, methotrexate, basiliximab, daclizumab, rituximab, anti-thymocyte globulin, anti-lymphocyte globulin, and a combination thereof.

A pharmaceutical composition containing a compound of the invention, although they are different by a kind of the object disease, may be used in combination with other medicines. Examples of the other medicines include, HDL-increasing drugs [squalene synthase inhibitor, CETP inhibitor, LPL activator, etc.]; preventive and therapeutic drug for HIV infectious disease [nucleic acid reverse transcriptase inhibitors such as tenofovir, tenofovir disoproxil or its fumarate salt, tenofovir alafenamide or its fumarate salt, zidovudine, didanosine, zalcitabine, lamivudine, stavudine, abacavir, fozivudine tidoxil, etc., non-nucleic acid reverse transcriptase inhibitors such as nevirapine, delavirdine, efavirenz, loviride, and etc., protease inhibitors such as atazanavir, darunavir, saquinavir, ritonavir, indinavir, nelfinavir, (fos)amprenavir, palinavir, lopinavir, etc.]; HMG-CoA reductase inhibitors [cerivastatin, atorvastatin, pravastatin, simvastatin, Itavastatin, lovastatin, (+)-3R,5S-7-[4-[4-fluorophenyl]-6-isopropyl-2-(N-methyl-N-methanesulfonylamino]pyrimidin-5-yl]-3,5-dihydroxy-6(E)-peptenoic acid, etc.]; atopic dermatitis drugs [sodium cromoglicate, etc.]; allergic nasal catarrh drugs [sodium cromoglicate, chlorpheniramine maleate, alimemazine tartrate, clemastine fumarate, homochlorcyclizine hydrochloride, terfenadine, mequitazine, etc.]; imipenem-cilastatin sodium; endotoxin antagonists or antibodies; oxidosqualene-lanosterol cyclase [e.g., decalin derivatives, azadecalin derivatives and indan derivatives]; calcium antagonists (diltiazem, etc.); glycerol; cholinesterase inhibitors (e.g., Aricept (donepezil), etc.); compounds suppressing cholesterol uptake [e.g., sitosterol, neomycin, etc.]; compounds inhibiting cholesterol biosynthses [e.g., HMG-CoA reductase inhibitors such as lovastatin, simvastatin, pravastatin, etc.]; cyclooxygenase depressants [Cox-I, Cox-II depressants such as celecoxib, rofecoxib, salicylic acid derivatives such as aspirin and the like, diclofenac, indometacin, loxoprofen, etc.]; sigal transduction inhibitors, squalene epoxidase inhibitors [e.g., NB-598 and the analogous compounds, etc.]; steroidal drugs [dexamethasone, hexestrol, methimazole, betamethasone, triamcinolone, triamcinolone acetonide, fluocinonide, fluocinolone acetonide, prednisolone, methylprednisolone, cortisone acetate, hydrocortisone, fluorometholone, beclomethasone dipropionate, estriol, etc.]; diacerin; nicotinic acid and derivatives and analogues thereof [e.g., acipimox and probucol]; nicergoline, nephrotic syndrome drugs: prednisolone (Predonine), prednisolone sodium succinate (Predonine), methylprednisolone sodium succinate (Solumedrol), betamethasone (Rinderon), dipyridamole (Persantine), dilazep dihydrochloride (Comelian), ticlopidine, clopidogrel, antiplatelet drugs and anticoagulants such as FXa inhibitors, etc.; barbital-based anticonulsants or anaesthetic drugs (phenobarbital, mephobarbital, metharbital, etc.); Parkinson disease drugs (e.g., L-DOPA, etc.); histamine receptor blockers (cimetidine, famotidine, etc.); hidantoin-based anticonvulsant drugs (phenyloin, mephenyloin, ethotoin, etc.); hydroxicam, fibrates [e.g., clofibrate, benzafibrate, gemfibrozil, etc.]; prostaglandins; megestrol acetate; gastric and intraduodenal ulcer drugs: antacids [e.g., histamine H2 antagonists (cimetidine, etc.), proton pump inhibitors (lansoprazole etc.,), etc.]; inflammatory mediator depressants; coronary vasodilators: nifedipine, diltiazem, nicorandil, nitrite drugs, etc.; infectious disease drugs: [e.g., antibiotic formulations (cefotiam hydrochloride, cefozopran hydrochloride, ampicillin, etc.), chemotherapeutic agents (sulfa drugs, synthetic antibacterial agents, antiviral agents, etc.), biologic formulations (vaccines, blood preparations including immunoglobulins) etc.] etc.; hepatic disease drugs: glycyrrhizin formulations [e.g., Stronger Minophagen, etc.]; liver hydrolysate; SH compounds [e.g., glutathione, etc.]; special amino acid formulations [e.g., amino]eban, etc.]; phospholipids [e.g., polyene-phosphatidyl choline, etc.]; vitamins [e.g., vitamin B1, B2, B6, B12, C, etc.]; adrenocortical hormones [e.g., dexamethasone, betamethasone, etc.]; interferons [e.g., interferon α, β, etc.]; hepatic encephalopathydrugs [e.g., lactulose, etc.]; hemostats used in cases of rapture of esophageal or gastricvenous cancer [e.g., vasopressin, somatostatin, etc] etc.; arthritis drugs; muscle relaxants [pridinol, tubocurarine, pancuronium, tolperisone hydrochloride, chlorphenesin carbamate, baclofen, chlormezanone, mephenesin, chlorzoxazone, eperisone, tizanidine, etc.]; vasodilators [oxyfedrine, diltiazem, tolazoline, hexobendine, bamethan, clonidine, methyldopa, guanabenz, etc.]; vasoconstrictors [dopamine, dobutamine denopamine, etc.]; antiplatelet drugs (ozagrel, etc.); thrombogenesis preventive and therapeutic drugs: anticoagulant drugs [e.g., heparin sodium, heparin calcium, warfarin calcium (Warfarin), Xa inhibitor]; thrombolytic drugs [e.g., tPA, urokinase]; antiplatelet drugs [e.g., aspirin, sulfinpyrazone (Anturan), dipyridamole (Persantine), ticlopidine (Panaldine), cilostazol (Pletaal), GPIIb/IIIa antagonist (ReoPro)]; antidepressants [imipramine, clomipramine, noxiptiline, feneridine, amitriptyline hydrochloride, nortriptyline hydrochloride, amoxapine, mianserin hydrochloride, maprotiline hydrochloride, sulpiride, fluvoxamine maleate, trazodone hydrochloride, etc.]; antiepileptic drugs [gavapentin, phenyloin, ethosuximide, acetazolamide, chlordiazepoxide, trimethadione, carbamazepine, phenobarbital, primidone, sultiame, sodium valproate, clonazepam, diazepam, nitrazepam, etc.]; antiallergic drugs [diphenhydramine, chlorpheniramine, tripelennamine, metodiramine, clemizole, diphenylpyraline, methoxyphenamine, sodium cromoglicate, tranilast, repininast, amlexanox, ibudilast, ketotifen, terfenadine, mequitazine, azlastin, epinastine, ozagrel hydrochloride, pranlukast hydrate, seratrodast, fexofenadine, ebastine, bucillamine, oxatomide, Stronger Neo-Minophagen C, tranexamic acid, ketotifen fumarate, etc.]; anticholinergic drugs (e.g., ipratropium bromide, flutropium bromide, oxitropium bromide, etc.); anti-Parkinson drugs (dopamine, levodopa, etc.); antirheumatic drugs; anti-inflammatory drugs (e.g., aspirin, acetaminophen, diclofenac sodium, ibuprofen, indometacin, loxoprofen sodium, dexamethasone, etc.); anticoagulant and antiplatelet drugs [sodium citrate, activated protein C, tissue factor pathway inhibitors, antithrombin III, dalteparin sodium, argatroban, gabexate, ozagrel sodium, ethyl icosapentate, beraprost sodium, alprostadil, pentoxifylline, tisokinase, streptokinase, heparin, etc.]; anticoagulant therapeutic drugs [dipyridamole (Persantine), dilazep hydrochloride (Comelian), ticlopidine, clopidogrel, Xa inhibitors]; antibacterial drugs [(1) sulfa drugs [sulfamethizole, sulfisoxazole, sulfamonomethoxine, sulfamethizole, salazosulfapyridine, sulfadiazine silver, etc.], (2) quinolone-based antibacterial drugs [nalidixic acid, pipemidic acid trihydrate, enoxacin, norfloxacin, ofloxacin, tosufloxacin tosilate, ciprofloxacin hydrochloride, lomefloxacin hydrochloride, sparfloxacin, fleroxacin, etc.], (3) antituberculous drugs [isoniazid, ethambutol (ethambutol hydrochloric acid), p-aminosalicyclic acid (calcium p-aminosalicylate), pyrazinamide, ethionamide, prothionamide, rifampicin, streptomycin sulfate, kanamycin sulfate, cycloserine, etc.], (4) anti-acid fast bacteria drugs [diaphenylsulfone, rifampicin, etc.], (5) antiviral drugs [idoxuridine, aciclovir, vidarabine, ganciclovir, etc.], (6) anti-HIV drugs [zidovudine, didanosine, zalcitabine, indinavir sulfate ethanolate, ritonavir, etc.], (7) spirocheticide, (8) antibiotics [tetracycline hydrochloride, ampicillin, piperacillin, gentamicin, dibekacin, kanendomycin, rokitamycin, tobramycin, amikacin, fradiomycin, sisomicin, tetracycline, oxytetracycline, rolitetracycline, doxycycline, ampicillin, piperacillin, ticarcillin, cephalothin, cephapirin, cephaloridine, cefaclor, cefalexin, cefroxadine, cefadroxil, cefamandole, cefotiam, cefuroxime, cefotiam, cefotiam hexetil, cefuroxime axetil, cefdinir, cefditoren pivoxil, ceftazidime, cefpiramide, cefsulodin, cefmenoxime, cefpodoxime proxetil, cefpirome, cefozopran, cefepime, cefsulodin, cefmetazole, cefminox, cefoxitin, cefbuperazone, latamoxef, flomoxef, cefazolin, cefotaxime, cefoperazone, ceftizoxime, moxalactam, thienamycin, sufazecin, aztreonam or salts thereof, griseofulvin, lankacidins [J. Antibiotics, 38, 877-885 (1985)], etc., cefixime, levofloxacin]; antithrombotic drugs (argatroban, etc.); antiprotozoal drugs [metronidazole, tinidazole, diethylcarbamazine citrate, quinine hydrochloride, quinine sulfate, etc.]; antitumor drugs [6-O—(N-chloroacetylcarbamoyl]fumagillol, bleomycin, methotrexate, actinomycin D, mitomycin C, daunorubicin, adriamycin, neocarzinostatin, cytosine arabinoside, fluorouracil, tetrahydrofuryl-5-fluorouracil, picibanil, lentinan, levamisole, bestatin, azimexon, glycyrrhizin, doxorubicin hydrochloride, aclarubicin hydrochloride, bleomycin hydrochloride, peplomycin sulfate, vincristine sulfate, vinblastine sulfate, irinotecan hydrochloride, cyclophosphamide, melphalan, busulfan, thiotepa, procarbazine hydrochloride, cisplatin, azathiopurine, mercaptopurine, tegafur, carmofur, cytarabine, methyltestosterone, testosterone propionate, testosterone enanthate, mepitiostane, fosfestrol, chlormadinone acetate, leuproline acetate, buserelin acetate, etc.]; antifungal drugs [(1) polyethylene-based antibiotics (e.g., amphotericin B, nystatin, trichomycin), (2) griseofulvin, pyrrolnitrin, etc., (3) cytosine metabolism antagonists (e.g., flucytosine), (4) imidazole derivatives (e.g., econazole, clotrimazole, miconazole nitrate, bifonazole, croconazole), (5) triazole derivatives (e.g., fluconazole, itoraconazole, azole compounds [2-[(1R,2R)-2-(2,4-difluorophenyl)-2-hydroxy-1-methyl-3-(1H-1,2,4-triazole-1-yl)propyl]-4-[4-(2,2,3,3-tetrafluoropropoxy)phenyl-3-(2H,4H)-1,2,4-triazolone], (6) thiocarbamate derivatives [e.g., trinaphthol], (7) echinocandin-based derivatives (e.g., caspofungin, FK-463, V-echinocandin), etc.]; antipsychotic drugs [chlorpromazine hydrochloride, prochlorperazine, trifluoperazine, thioridazine hydrochloride, perphenazine maleate, fluphenazine enanthate, prochlorperazine maleate, levomepromazine maleate, promethazine hydrochloride, haloperidol, bromperidol, spiperone, reserpine, clocapramine hydrochloride, sulpiride, zotepine, etc.]; antiulcer drugs [metoclopramide, histidine hydrochloride, lansoprazole, metoclopramide, pirenzepine, cimetidine, ranitidine, famotidine, urogastron, oxethazaine, proglumide, omeprazole, sucralfate, sulpiride, cetraxate, gefarnate, aldioxa, teprenone, prostaglandins etc.]; anti diabetic drugs [e.g., pioglitazone, nateglinide, voglibose, acarbose, etc.]; antiobese drugs [mazindol, etc.]; antirheumatic drugs; antianxiety drugs [diazepam, lorazepam, oxazepam, chlordiazepoxide, medazepam, oxazolam, cloxazolam, clotiazepam, bromazepam, etizolam, fludiazepam, hydroxyzine, etc.]; antiarrhythmic drugs [disopyramide, lidocaine, quinidine sulfate, flecamide acetate, mexiletine hydrochloride, amiodarone hydrochloride, and β blockers, Cα antagonists, etc.; antiasthmatic drugs [isoprenaline hydrochloride, salbutamol sulfate, procaterol hydrochloride, terbutaline sulfate, trimetoquinol hydrochloride, tulobuterol hydrochloride, orciprenaline sulfate, fenoterol hydrobromide, ephedrine hydrochloride, ipratropium bromide, oxitropium bromide, flutropium bromide, theophylline, aminophylline, sodium cromoglicate, tranilast, repirinast, amlexanox, ibudilast, ketotifen, terfenadine, mequitazine, azelastine, epinastine, ozagrel hydrochloride, pranlukast hydrate, seratrodast, dexamethasone, prednisolone, hydrocortisone, beclomethasone propionate, fluticasone propionate, beclomethasone propionate, procaterol, etc.]; anti-hypothyroidism drugs [dried thyroid (Thyreoid), levothyroxine sodium (Tyradin S), liothyronine sodium (thyronine, tyronamine)]; nephrotic syndrome drugs [prednisolone (Predonine), prednisolone sodium succinate (Predonine), methylprednisolone sodium succinate (Solumedrol), betamethasone (Rinderon)]; antihypertensive drugs {(1) sympathetic nerve depressants [α2 stimulating drugs (e.g., clonidine, guanabenz, guanfacine, methyldopa, etc.), ganglionic blockers (e.g., hexamethonium, trimethaphan, etc.), presynaptic blockers (e.g., Alusa-Oxylone, dimethylamino reseru pinate, rescinnamine, reserpine syrosingopine, etc.), neuronal blockers (e.g., betanidine guanethidine, etc.), α1 blockers (e.g., bunazosin, doxazosin, prazosin, terazosin, urapidil, etc.), [3 blockers (e.g., propranolol, nadolol, timolol, nipladilol, bunitrolol, indenolol, penbutolol, carteolol, carvedilol, pindolol, acebutolol, atenolol, bisoprolol, metoprolol, labetalol, amosulalol, arotinolol, etc.), etc], (2) vasodilators [calcium channel antagonists (e.g., manidipine, nicardipine, nilvadipine, nisoldipine, nitrendipine, benidipine, amlodipine, aranidipine, etc.), phthalazine derivatives (e.g., budralazine, cadralazine, ecarazine, hydralazine, todralazine, etc.), etc.], (3) ACE inhibitors [alacepril, captopril, cilazapril, delapril, enalapril, lisinopril, temocapril, trandolapril, quinapril, imidapril, benazepril, perindopril, etc.)], (4) AII antagonists [losartan, candesartan, valsartan, telmisartan, irbesartan, forasartan, etc.], (5) diuretic drugs [e.g., diuretic drugs described above, etc.]}; antihypertensive drugs {diuretic drugs [e.g., furosemide (Lasix), bumetanide (Lunetoron), azosemide (DIART)], antihypertensive drugs [e.g., ACE inhibitors, (enalapril maleate (RENIVACE) etc.,) and Cα antagonists (manidipine, amlodipine. etc.), α or β receptor blockers, etc.], antihyperlipemia drugs [HMG-CoA reductase inhibitors (e.g., lvastatin, cerivastatin, atorvastatin, etc.), fibrates [e.g., simfibrate, aluminum clofibrate, clinofibrate, fenofibrate, etc.], anion exchange resin [e.g., cholestyramine, etc.], nicotinic acid drugs [e.g., nicomol, niceritrol, tocopherol nicotinate etc.], polyvalent unsaturated fatty acid derivatives [e.g., ethyl icosapentaenoic acid, polyene phosphatidyl choline, melinamide, etc.], phytosterols [e.g., .gamma.-oryzanol, soy sterol, etc.], elastase, sodium dextran sulfate, squalene synthase inhibitors, CETP inhibitors, 2-chloro-3-[4-(2-methyl-2-phenylpropoxy)phenyl]ethyl propionate [Chem. Pharm. Bull., 38, 2792-2796 (1990)], etc.}; osseous disease drugs {calcium formulations [e.g., calcium carbonate, etc.], calcitonin formulations, activated vitamin D3 formulations [e.g., alfacalcidol (Alfarol etc.), calcitriol (ROCALTROL), etc.], sex hormones [e.g., estrogen, estradiol, etc.], hormone formulations [e.g., conjugated estrogen (Premarin), etc.], ipriflavone formulations [osten, etc.], vitamin K2 vitamin K2 formulations [e.g., menatetrenone (Glakay), etc.], bis-phosphonate-based formulations [etidronate, etc.], prostaglandin E2, fluorine compounds [e.g., sodium fluoride, etc.], bone morphogenetic protein (BMP), fibroblast growth factor (FGF), platelet derived growth factor (PDGF), transforming growth factor (TGF-β), insulin-like growth factor-1 and -2 (IGF-1, -2), parathyroid adrenal hormones (PTH), and compounds described in EP-A1-376197, EP-A1-460488, and EP-A1-719782 [e.g., (2R,4S)-(−)-N-[4-(diethoxyphosphorylmethyl)phenyl]-1,2,4,5-tetrahydro-4-methyl-7, 8-methylenedioxy-5-oxo-3-bemzothiepin-2-carboxamide, etc.], etc.), lipid-soluble vitamin drugs (1) vitamin A family (vitamin A1, vitamin A2, and retinol palmitate), (2) vitamin D family (vitamin D1, D2, D3, D4 and D5), (3) vitamin E family (α-tocopherol, β-tocopherol, γ-tocopherol, δ-tocopherol, dl-α-tocopherol nicotinate.), (4) vitamin K family (vitamin K1, K2, K3 and K4), (5) folic acids (vitamin M), etc.]; vitamin derivatives [various vitamin derivatives, e.g., vitamin D3 derivatives such as 5,6-trans-cholecalciferol, 2,5-hydroxycholecalciferol, 1-α-hydroxycholecalciferol, vitamin D2 derivatives such as 5,6-trans-ergocalciferol, and the like]; disease-modifying antirheumatic and immunosuppressive drugs [e.g., methotrexate, leflunomide, prograf, sulfasalazine, D-penicillamine, the oral gold salts]; hypertensors [dopamine, dobutamine, denopamine, digitoxin, digoxin, methyldigoxin, lanatoside C, G-strophanthin, etc.]; myocardial protective drugs: heart ATP-K opener (Na—H exchange inhibitors, endothelin antagonists, urotensin antagonist, etc.), cardiac failure drugs [cardiac stimulants (e.g., digitoxin, digoxin, methyldigoxin, lanatoside C, proscillaridin, etc.), α, β stimulating drugs (e.g., epinephrine, norepinephrine, isoproterenol, dopamine, docarpamine, dobutamine, denopamine, etc.), phosphodiesterase inhibitors (e.g., aminone, milrinone, olprinone hydrochloride, etc.), calcium channel sensibility improvers (e.g., pimobendan, etc.), nitrate drugs (e.g., nitroglycerin, isosorbide nitrate, etc.), ACE inhibitors (e.g., the ACE inhibitor described above, etc.), diuretic drugs (e.g., diuretic drugs described above, etc.), calperitide, ubidecarenone, vesnarinone, aminophylline, etc.]; neurotrophic factors; renal failure and nephropathia drugs; biologic formulations [e.g., monoclonal antibodies (e.g., anti-TNF-α antibodies anti-IL-12 antibodies, anti-IL-6 antibodies, anti-ICAM-1 antibodies, anti-CD4 antibodies, etc.), soluble receptors (e.g., soluble TNF-α receptors, etc.), protein ligands (IL-1 receptor antagonist, etc.)]; bile acid binding resins [e.g., cholestyramine, cholestipol, etc.]; biliary tract disease drugs:cholepoietic drugs [e.g., dehydrocholic acid, etc.], cholekinetic drugs [e.g., magnesium sulfate, etc.], etc.; central nervous system agonists:antianxiety drugs, hypnotic and sedative drugs, anesthetic drugs, spasmolytic drugs, autonomic drugs, anti-Parkinson drugs and other psychoneuro drugs, etc.; antitiussive and expectorants [ephedrine hydrochloride, noscapine hydrochloride, codeine phosphate, dihydrocodeine phosphate, isoproterenol hydrochloride, ephedrine hydrochloride, methylephedrine hydrochloride, noscapin hydrochloride, arocloramide, chlofedanol, picoperidamine, cloperastine, protoxlol, isoproterenol, salbutamol, terbutaline, oxymetebanol, morphine hydrochloride, dextromethorphan hydrobromide, oxycodone hydrochloride, dimemorfan phosphate, tipepidine hibenzate, pentoxyverine citrate, clofedanol hydrochloride, benzonatate, guaifenesin, bromhexine hydrochloride, ambroxol hydrochloride, acetylcysteine, ethylcysteine hydrochloride, carbocisteine, etc.], sedative drug [chlorpromazine hydrochloride, atropine sulfate, phenobarbital, barbital, amobarbital, pentobarbital, thiopental sodium, thiamylal sodium, nitrazepam, estazolam, flurazepam, haloxazolam, triazolam, flunitrazepam, bromovalerylurea, chloral hydrate, triclofos sodium, etc.], analgesic and antiphlogistic drugs [e.g., central analgesic drugs (e.g., morphine, codeine, pentazocine etc.), steroid drugs (e.g., prednisolone, dexamethasone, betamethasone), etc., antiphlogistic enzymic drugs (e.g., bromersine, lysozymes, protease, etc.)], diabetic drugs [sulfonylurea drugs (e.g., tolbutamide, chlorpropamide, glyclopyramide, acetohexamide, tolazamide, glibenclamide, glybuzole, etc.), biguanide drugs (e.g., metformin hydrochloride, buformin hydrochloride, etc.), α-glucosidase inhibitors (e.g., voglibose, acarbose, etc.), insulin resistance improvers (e.g., pioglitazone, troglytazone, etc.), insulin, glucagon, diabetic complication drugs (e.g., epalrestat, thioctic acid, etc.), actos, rosiglatazone, kinedak, penfill, humulin, euglucon, glimicron, daonil, novolin, monotard, insulin family, glucobay, dimelin, rastinone, bacilcon, deamelin S, Iszilin family, etc.]; brain function diluting agents (e.g., idebenone, vinpocetin, etc.); urinary and mele genital disease drugs [e.g., prostatomegaly drugs (tamsulosin hydrochloride, prazosin hydrochloride, chlormadinone acetate, etc.), prostate cancer drugs (leuprorelin acetate, goserelin acetate, chlormadinone acetate, etc.)], etc; nonsteroidal antiinflammatory drugs [acetaminophen, phenacetin, ethenzamide, sulpyrine, antipyrine, migrenin, aspirin, mefenamic acid, fulfenamic acid, diclofenac sodium, loxoprofen sodium, phenylbutazone, indomethacin, ibuprofenn, ketoprofen, naproxen, oxaoprozin, flurbiprofen, fenbufen, pranoprofen, floctafenine, epirizole, tiaramide hydrochloride, zaltoprofen, gabexate mesilate, camostat mesilate, urinastatin, colchicine, probenecid, sulfinpyrazone, benzbromarone, allopurinol, sodium aurothiomalate, sodium hyaluronate, sodium salicylate, morphine hydrochloride, salicyclic acid, atropine, scopolamine, morphine, pethidine, levorphanol, ketoprofen, naproxen, oxymorphine or the salts thereof, etc.]; frequent urination and anischuria drugs [flavoxate hydrochloride, etc.]; unstable plaque stablizers [MMP inhibitors, chymase inhibitors, etc.]; arrhythmic drugs [sodium channel blockers (e.g., quinidine, procainamide, disopyramide, ajmaline, cibenzoline, lidocaine, diphenylhydantoin, mexiletine, propafenone, flecamide, pilsicamide, phenyloin, etc.), β blockers (e.g., propranolol, alprenolol, bufetolol, oxprenolol, atenolol, acebutolol, metoprolol, bisoprolol, pindolol, carteolol, arotinolol, etc.), potassium channel blockers (e.g., amiodarone, etc.), calcium channel blockers (e.g., verapamil, diltiazem, etc.), etc.]; gynecologic disease drugs [e.g., climacteric disorder drugs (conjugated estrogen, estradiol, testosterone enanthate, valerate estradilo, etc.), breast cancer drugs (tamoxifen citrate, etc.), endometriosis and hysteromyoma drugs (leuprorelin acetate, danazol, etc.)], etc.; anesthetic drugs [a. local anaesthetic drugs [cocaine hydrochloride, procaine hydrochloride, lidocaine, dibucaine hydrochloride, tetracaine hydrochloride, mepivacaine hydrochloride, bupivacaine hydrochloride, ocybuprocaine hydrochloride, ethyl aminobenzoate, oxethazaine], etc.]; b. general anesthetic drugs [(1) inhalation anesthetic drugs (e.g., ether, halothane, nitrous oxide, influrane, enflurane), (2) intravenous anesthetic drugs (e.g., ketamine hydrochloride, droperidol, thiopental sodium, thiamylal sodium, pentobarbital), etc.]]; anesthetic antagonists [levallorphan, nalorphine, naloxone, or the salts thereof, etc.]; chronic cardiac failure drugs: cardiac stimulants [e.g., cardiac glycoside (digoxin), etc., β receptor stimulating drugs (catecholamine preparations such as denopamine, dobutamine.), PDE inhibitors, etc.]; diuretic drugs [e.g., furosemide (Lasix), spironolactone (Aldactone), bumetanide (Lunetoron), azosemide (Diart), etc.]; ACE inhibitors [e.g., enalapril maleate (Renivace), etc.]; Ca antagonists [e.g., amlodipine, manidipine, etc.] and β receptor blockers, etc.; immunomodulators [cyclosporin, tacrolimus, gusperimus, azathioprine, antilymphocyte sera, dried sulfonated immunoglobulins, erythropoietins, colony stimulating factors, interleukins, interferons, etc.]; diuretic drugs [thiazide-based diuretic drugs (benzylhydrochlorothiazide, cyclopenthiazide, ethiazide, hydrochlorothiazide, hydroflumethiazide, methylclothiazide, penflutiazide, polythiazide, trichlormethiazide, etc.), loop diuretic drugs (chlortalidone, clofenamide, indapamide, mefruside, meticrane, sotrazone, tripamide, quinethazone, metolazone, furosemide, mefruside, etc.), potassium-sparing diuretic drugs (spironolactone, triamterene, etc.)]; and erectile dysfunction drugs (Viagra, apomorphine, etc.).

These drugs, separately or simultaneously may be prepared by mixing with pharmaceutically carriers, excipients, binders, diluents or the like which can be accepted pharmacologically, and can be administered either orally or parenterally. When the drug is prepared separately, the drugs which are prepared separately may be mixed with a diluent or the like before using and then administered, or each of the preparations separately prepared may be administered, simultaneously or separately at an interval, to an identical person. Kit products used for mixing the separately-prepared preparations with a diluent and the like before using and administering, (for example, an injectable kit including ampoules for containing each powdery drug, and a diluent for mixing and solving with 2 or more drugs before using, and the like), kit products used for administering each of the separately-prepared preparation, formulation, simultaneously or separately at an interval, to an identical person, (for example, a tablet kit for 2 or more tablets, simultaneously or separately at an interval, put the tablet which is contained each drugs into the same or the separate bags, if necessary, a column provided on the bags wherein the drug administration date is to be indicated, and the like), or the like are also included in the pharmaceutical composition of the present invention.

The following Examples further illustrate the present invention in detail but are not to be construed to limit the scope thereof.

### Examples

### Example 1—Cenicriviroc Mesylate Compositions

A series of cenicriviroc mesylate compositions that were identical except for the identity of the acid solubilizer were prepared by wet granulation in a Key 1L bowl granulator, followed by tray drying, sieving, mixing and compression into tablets on a Carver press. The composition of the formulations is shown in Table 1.

TABLE 1

| | Unit Formula (mg/unit) | | | |
|---|---|---|---|---|
| | Ex. 1a | Ex. 1b | Ex. 1c | Ex. 1d |
| | Citric | Fumaric | Maleic | Sodium |
| Components | Acid | Acid | Acid | Bisulfate |
| Cenicriviroc Mesylate | 28.45 | 28.45 | 28.45 | 28.45 |
| Mannitol | 7.88 | 7.88 | 7.88 | 7.88 |
| Hydroxypropyl | 2.62 | 2.62 | 2.62 | 2.62 |
| Cellulose | | | | |
| Croscarmellose Sodium | 1.75 | 1.75 | 1.75 | 1.75 |
| Croscarmellose Sodium | 1.75 | 1.75 | 1.75 | 1.75 |
| Citric Acid | 43.75 | — | — | — |
| Fumaric Acid | — | 43.75 | — | — |
| Maleic Acid | — | — | 43.75 | — |
| Sodium Bisulfate | — | — | — | 43.75 |
| Silicon Dioxide | 0.43 | 0.43 | 0.43 | 0.43 |
| Magnesium Stearate | 0.88 | 0.88 | 0.88 | 0.88 |
| Total | 87.5 | 87.5 | 87.5 | 87.5 |

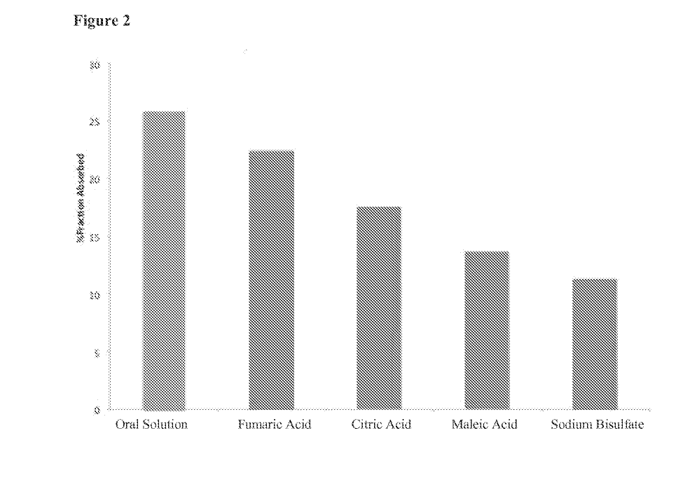

The tablets were administered to beagle dogs. An oral solution was also administered as a control. The absolute bioavailabilities of the formulations and of the oral solution were determined, and are shown in FIG. 2. The result shows that the cenicriviroc mesylate with fumaric acid has a significantly higher bioavailability than any of the other solubilizers tested.

### Example 2: Cenicriviroc Mesylate Compositions

Cenicriviroc mesylate, fumaric acid, microcrystalline cellulose, cross-linked sodium carboxymethyl cellulose, and magnesium stearate were admixed, dry granulated, milled, blended with extragranular microcrystalline cellulose, cross-linked sodium carboxymethyl cellulose, and magnesium stearate and compressed into tablets having a hardness greater than 10 kP and friability less than 0.8% w/w. The resulting tablets had the composition shown in Table 2.

TABLE 2

| | Unit Formula (mg/unit) | | | | |
|---|---|---|---|---|---|
| Components | Ex. 2a | Ex. 2b | Ex. 2c | Ex. 2d | Ex. 2e |
| Cenicriviroc Mesylate | 170.69^{a} | 170.69^{a} | 170.69^{a} | 170.69^{a} | 170.69^{a} |
| Fumaric Acid | 160.00 | 160.00 | 160.00^{b} | 160.00 | 80.00 |
| Microcrystalline | 252.68 | 272.18 | 272.18 | 272.18 | 66.35 |
| Cellulose | | | | | |
| Crospovidone | — | — | — | 19.50 | — |
| Croscarmellose Sodium | 58.50 | 39.00 | 39.00 | 19.50 | 20.70 |
| Magnesium Stearate | 8.13 | 8.13 | 8.13 | 8.13 | 2.55 |
| Total | 650.0 | 650.0 | 650.0 | 650.0 | 340.0 |

^{a}Equivalent to 150 mg cenicriviroc freebase.
^{b}Added in the extragranular portion of the powder blend.

By way of illustration, the concentration percentage and mass per tablet of the components in Example 2b (i.e., Ex. 2b) are given in Table 3.

TABLE 3

| | Concentration | Mass (mg) per |
|---|---|---|
| Component | (% w/w) | tablet |
| Cenicriviroc mesylate | 26.26 | 170.69^{a} |
| Fumaric acid | 24.62 | 160.00 |
| Microcrystalline cellulose | 41.87 | 272.18 |
| Cross-linked sodium | 6.00 | 39.00 |
| carboxymethyl cellulose | | |
| Magnesium stearate | 1.25 | 8.13 |
| Total | 100.0 | 650.0 |

^{a}equivalent to 150 mg cenicriviroc free base

### Example 3: Cenicriviroc Mesylate Compositions

Cenicriviroc mesylate, microcrystalline cellulose, cross-linked sodium carboxymethyl cellulose, and magnesium stearate were admixed, dry granulated, dried, milled, blended with extragranular microcrystalline cellulose, cross-linked sodium carboxymethyl cellulose, fumaric acid, colloidal silicon dioxide, and magnesium stearate and compressed into tablets having a hardness greater than 10 kP and friability less than 0.8% w/w. The resulting tablets had the composition shown in Table 4.

TABLE 4

| | Concentration | Mass (mg) per |
|---|---|---|
| Component | (% w/w) | tablet |
| Cenicriviroc mesylate | 26.26 | 28.45^{a} |
| Fumaric acid | 24.62 | 26.67 |
| Microcrystalline cellulose | 41.87 | 45.36 |
| Cross-linked sodium | 6.00 | 39.00 |
| carboxymethyl cellulose | | |
| Magnesium stearate | 1.25 | 1.35 |
| Total | 100.0 | 108.3 |

^{a}equivalent to 25 mg cenicriviroc free base

Notably, the formulation of Table 4 has the same ratio of components as that of Table 3, and differs only in the total amount of the components that are used for each tablet. Thus, Table 3 shows tablets with 150 mg cenicriviroc (based on free base), whereas Table 4 shows tablets with 25 mg cenicriviroc (based on free base) with the same ratio of components as the 150 mg tablets of Example 2b, shown in Table 3.

### Example 4—Reference

The citric acid based formulation of Table 5 was prepared as follows. Cenicriviroc, hydroxypropyl cellulose, mannitol, and cross-linked sodium carboxymethyl cellulose were admixed, wet granulated, dried, milled, and blended with microcrystalline cellulose, cross-linked sodium carboxymethyl cellulose, citric acid, colloidal silicon dioxide, talc, and magnesium stearate. The resulting blend was compressed into tablets having a hardness greater than 10 kP and friability less than 0.8% w/w. The tablets were coated with hydroxypropyl methylcellulose, polyethylene glycol 8000, titanium dioxide, and yellow iron oxide. The coated tablets thus produced were substantially identical to those disclosed in U.S. Patent Application Publication No. 2008/031942 (see, e.g., Table 3).

TABLE 5

| Component | mg/tablet | % w/w |
|---|---|---|
| Cenicriviroc mesylate | 28.91 | 4.68 |
| Mannitol | 341.09 | 56.85 |
| Microcrystalline cellulose | 80.00 | 12.94 |
| Colloidal silicon dioxide | 12.00 | 2.00 |
| Citric acid anhydrous | 75.00 | 12.14 |
| Hydroxypropyl cellulose | 12.00 | 1.94 |
| Cross-linked sodium carboxymethyl cellulose | 30.00 | 4.85 |
| Talc | 12.00 | 1.94 |
| Magnesium stearate | 9.00 | 1.46 |
| Hydroxypropyl methylcellulose | 11.71 | 1.89 |
| Polyethylene glycol 8000 | 2.69 | 0.44 |
| Titanium dioxide | 3.03 | 0.49 |
| Yellow iron oxide | 0.57 | 0.09 |

### Example 5—Reference

Cenicriviroc and hypromellose acetate succinate were dissolved in methanol and spray dried into a fine powder containing 25% cenicriviroc by weight (based on the weight of cenicriviroc free base). The powder was admixed with colloidal silicon dioxide, microcrystalline cellulose, mannitol, sodium lauryl sulfate, cross-linked sodium carboxymethyl cellulose, and magnesium stearate. The admixture was compressed into tablets having a hardness greater than 10 kP and friability less than 0.8% w/w. The final composition of the tablets is shown in Table 6.

TABLE 6

| Component | Weight % | Mass (mg) |
|---|---|---|
| Cenicriviroc (as mesylate salt) | 8.33 | 50.00 |
| Hypromellose acetate succinate | 25.00 | 150.00 |
| Sodium lauryl sulfate | 2.00 | 12.00 |
| Cross-linked sodium carboxymethyl | 6.00 | 36.00 |
| cellulose | | |
| Microcrystalline cellulose | 27.83 | 167.00 |
| Mannitol | 27.83 | 167.00 |
| Colloidal silicon dioxide | 1.00 | 6.00 |
| Magnesium stearate | 2.00 | 12.00 |
| Total | 100.0 | 600.0 |

### Example 6: Bioavailibility of CVC Formulation

The absolute bioavailability of the tablets of Example 3 in beagle dogs was compared to that of the tablets of Examples 4 and 5, as well as to both an oral solution of cenicriviroc mesylate and a gelatin capsule containing cenicriviroc mesylate powder. The results are shown in Table 7.

TABLE 7

| Component | Absolute bioavailability(%) |
|---|---|
| Oral Solution | 25.8 |
| Powder in capsule | 6.4 |
| Example 3 | 26.6 |
| Example 4 | 21.1 |
| Example 5 | 12.4 |

This example demonstrates that the bioavailability of cenicriviroc in dry granulated tablets with fumaric acid (Ex. 3) is substantially similar to that of an oral solution, and is significantly higher than the bioavailability of cenicriviroc in wet granulated tablets with fumaric (Ex. 1) or citric acid (Ex. 4), and over double that of cenicriviroc in tablets with amorphous cenicriviroc in a spray dried dispersion with HPMC-AS (Ex. 5). These results are surprising, because there was no reason to suspect that dry granulation of crystalline API provides a significant increase in bioavailability over wet granulation and amorphous spray dried dispersions. This is especially so because amorphous spray dried dispersions are frequently used to increase the bioavailability of poorly water soluble drugs. These results are also surprising because fumaric acid has a slower dissolution time than citric acid and was used at a lower mass ratio of acid relative to CVC API (3:1 for citric acid:API versus 1.06:1 fumaric acid:API). Hence it was therefore surprising that fumaric acid proved to be a more effective solubilizer than citric acid for CVC.

### Example 7: Accelerated Stability of CVC Formulation

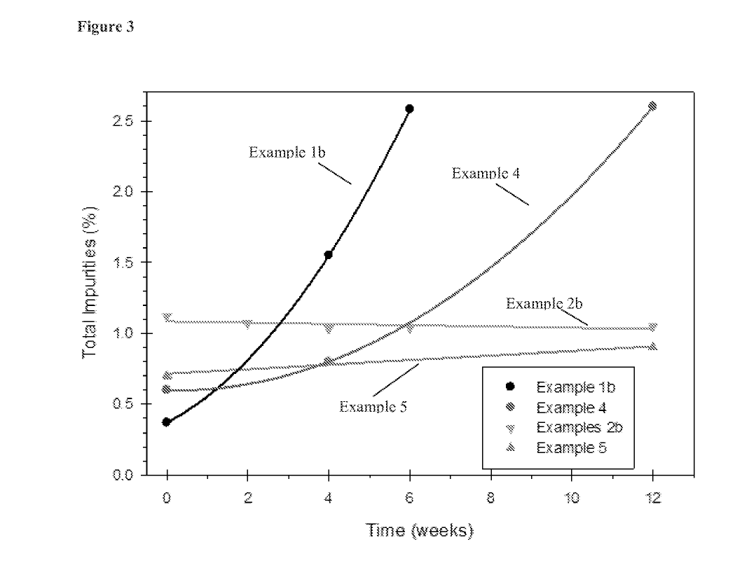

The accelerated stability of the tablets of Example 2b was compared to that of the tablets of Examples 1, 4, and 5 via exposure to an environment of 75% relative humidity at 40° C. All tablets were packaged with a desiccant during the study. As shown in FIG. 3, the tablets of Examples 2b are surprisingly much more stable than the other wet granulated tablets, and similarly stable as the spray dried dispersion tablets. This difference in stability between the tablets of Examples 2b and Example 4 is particularly surprising since the only significant difference between the two is the method of making the formulations (dry granulation vs. wet granulation). These results are also surprising, because it was not previously known that the method of granulation could have an effect on both cenicriviroc bioavailability and stability.

### Example 8: Stability of CVC Formulation

The stability of the tablets of Examples 2 and 3 was tested by exposing the tablets to an environment of 75% relative humidity at 40° C. for six weeks. All tablets were packaged with a desiccant during the study. The results are shown in Table 8, which shows that the tablets are very stable under these conditions.

TABLE 8

| Time (Weeks) | Water content (%) | Strength (%) | Total Impurities (%) |
|---|---|---|---|
| 0 | 1.5 | 99.1 | 1.2 |
| 2 | 1.4 | 99.2 | 1.1 |
| 4 | 1.4 | 98.0 | 1.0 |
| 6 | 1.4 | 98.6 | 1.0 |

### Example 9: Stability of CVC Formulations

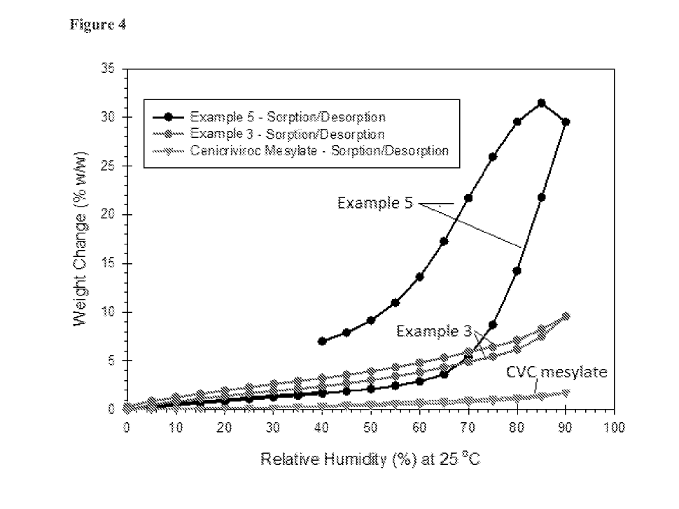

Dynamic vapor sorption isotherms at 25° C. correlate to the stability of the tablets of Examples 3 and 4 with that of cenicriviroc mesylate. Sorption was performed from 0% relative humidity to 90% relative humidity at 5% intervals. At each interval, each sample was equilibrated for no less than 10 minutes and no longer than 30 minutes. Equilibration was stopped when the rate of mass increase was no more than 0.03% w/w per minute or after 30 minutes, whichever was shorter. The result, which appears in FIG. 4, shows that tablets of Example 2b are significantly more stable than those of Example 4. This result is consistent with Example 3 being significantly less hygroscopic than Example 4. The increased hygroscopicity of Example 4, in comparison to Examples 2b, can be associated with a higher mobile water content which can in turn cause partial gelation and subsequent decreased stability of Example 4.

### Example 10: Bioavailability of CVC Formulations

The bioavailability of the tablets of Example 3 was compared to that of Example 5 and cenicriviroc mesylate powder in a gelatin capsule in different stomach states in beagle dogs. The bioavailability was tested under different pre-treatment states, each of which alters the gastric pH. Specifically, pentagastric pretreatment provides the lowest pH, no treatment provides an intermediate pH, and famotidine treatment provides the highest pH.

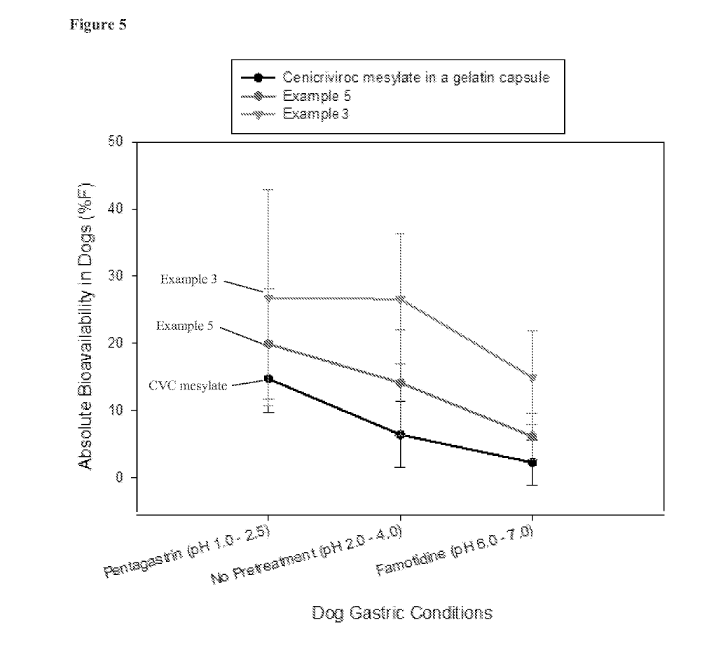

The result, which appears in FIG. 5, shows that the tablets of Example 3 has a higher bioavailability under all conditions that were tested. The bioavailability of Example 3 varied less between pentagastrin treated and untreated dogs, whereas Example 4 showed a significant loss of bioavailability in fasted, non-treated dogs (intermediate gastric pH) compared to that in pentagastrin treated dogs (lowest gastric pH). Pretreatment with famotidine, an H2 receptor agonist that suppresses stomach acidity and raises gastric pH decreased bioavailability for all samples, however, the reduction for Example 3 was much less than that for Example 4.

These results demonstrate an additional unexpected benefit of dry granulated cenicriviroc compositions with fumaric acid. Specifically, the pharmacokinetics of such formulations do not vary as much as those of the spray dried dispersion (Example 4) when administered across the full range of potential human gastric pH conditions. This result is unexpected and surprising, because the bioavailability of other weakly basic antiretroviral drugs, such as atazanavir, is greatly affected by the gastric pH. For such drugs, changes in gastric pH, which can be caused by a disease or medical condition, such as achlorohydric patients, or by co-administration of drugs such as antacids, proton pump inhibitors, or H2 receptor agonists, can lower the bioavailability to sub-therapeutic levels. These results showing that the dry granulated, fumaric acid based cenicriviroc mesylate formulation of Example 3 is less prone to bioavailability changes as the gastric pH changes shows that Example 3 is a more robust formulation that can be used in patients who have or are likely to have varying gastric pH levels.

### Examples 11a-11c: Preparation of Cenicriviroc Mesylate and Lamivudine Formulations

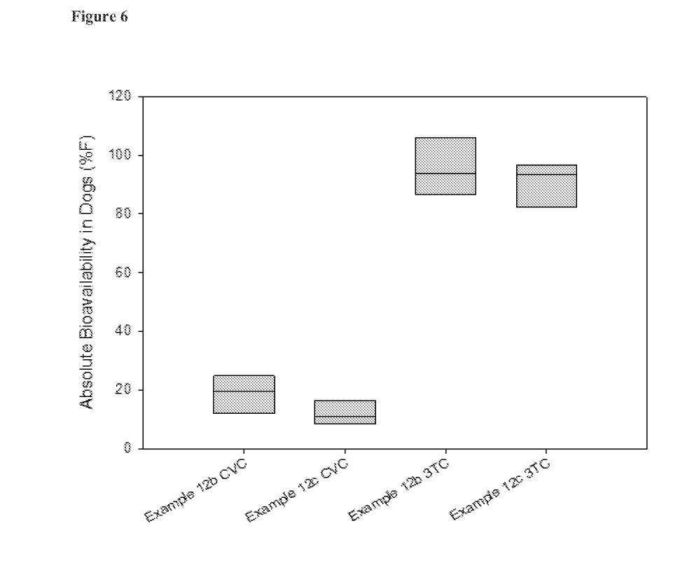

The formulations of cenicriviroc mesylate and lamivudine of Table 9 were prepared as follows. First, the intragranular components were admixed and dry granulated to form a composition as a dry granulated admixture. This dry granulated admixture was then further admixed with the extragranular components to form a mixture. The mixture was compressed into tablets. The absolute bioavailability of the cenicriviroc (CVC) and lamivudine (3TC) in beagle dogs in the 150 mg CVC strength tablets (Examples 11b and 11c) were measured. The results are shown in FIG. 6.

TABLE 9

| Example 12a | | Example 12b | | Example 12c | |
|---|---|---|---|---|---|
| 25 mg cenicriviroc | | 150 mg cenicriviroc | | 150 mg cenicriviroc | |
| and 300 mg | | and 300 mg | | and 300 mg | |
| lamivudine | | lamivudine | | lamivudine | |
| % w/w | mg/tablet | % w/w | mg/tablet | % w/w | mg/tablet |

Intragranular Components

| | | | | | | |
|---|---|---|---|---|---|---|
| Cenicriviroc mesylate | 5.69 | 28.45 | 17.97 | 170.69 | 21.34 | 170.69 |
| Fumaric Acid | 5.33 | 26.67 | 16.84 | 160.00 | 20.00 | 160.00 |
| Microcrystalline cellulose | 5.82 | 29.11 | 18.39 | 19.50 | 2.64 | 21.10 |
| Cross-linked sodium | 0.65 | 3.25 | 2.05 | 19.50 | 2.64 | 21.10 |
| carboxymethyl cellulose | | | | | | |
| Magnesium | 0.16 | 0.81 | 0.51 | 4.88 | 0.53 | 4.20 |
| stearate | | | | | | |

Extragranular Components

| | | | | | | |
|---|---|---|---|---|---|---|
| Lamivudine (3TC) | 60.00 | 300.00 | 31.58 | 600.00 | 37.50 | 300.00 |
| Microcrystalline cellulose | 16.34 | 81.71 | 6.39 | 60.75 | 3.78 | 30.21 |
| Cross-linked sodium | 5.00 | 25.00 | 5.26 | 50.00 | 5.00 | 40.00 |
| carboxymethyl cellulose | | | | | | |
| Magnesium stearate | 1.00 | 5.00 | 1.00 | 9.50 | 1.00 | 8.00 |
| Total per tablet | 100.00 | 500.00 | 100.00 | 950.00 | 100.00 | 800.00 |

### Example 12: Improvements in APRI and FIB-4 Fibrosis Scores Correlate with Decreases in sCD14 in HIV-1 Infected Adults Receiving Cenicriviroc Over 48 Weeks

Background:

Cenicriviroc (CVC), a novel, oral, once-daily CCR2/CCR5 antagonist, has demonstrated favorable safety and anti-HIV activity in clinical trials. CVC demonstrated antifibrotic activity in two animal models of liver disease. Post-hoc analyses were conducted on APRI and FIB-4 scores in Study 202 (NCT01338883).

Methodology:

143 adults with CCR5-tropic HIV-1, BMI≦35 kg/m² and no apparent liver disease (i.e., ALT/AST Grade≦2, total bilirubin≦ULN, no HBV, HCV, active or chronic liver disease, or cirrhosis) were randomized 4:1 to CVC or efavirenz (EFV). APRI and FIB-4 scores were calculated. Change in score category from baseline (BL) to Weeks 24 and 48 was assessed in patients with non-missing data. Correlations between changes from BL in APRI and FIB-4 scores, and MCP-1 (CCR2 ligand) and sCD14 (inflammatory biomarker) levels were evaluated.

TABLE

| | | CVC | | | EFV | | |
|---|---|---|---|---|---|---|---|
| | Fibrosis | Baseline | Week 24 | Week 48 | Baseline | Week 24 | Week 48 |
| | index | (n = 113) | (n = 92) | (n = 80) | (n = 28) | (n = 20) | (n = 17) |
| APRI category | <0.5 | 84% | 93% | 91% | 96% | 100% | 100% |
| | 0.5-1.5 | 14% | 7% | 8% | 4% | — | — |
| | >1.5 | 2% | — | 1% | — | — | — |
| | Decreased 1 | N/A | 14% | 10% | N/A | 5% | 6% |
| | category from | | | | | | |
| | baseline | | | | | | |
| FIB-4 category | <1.45 | 82% | 93% | 94% | 100% | 100% | 94% |
| | 1.45-3.25 | 17% | 7% | 5% | — | — | 6% |
| | >3.25 | 1% | — | 1% | — | — | — |
| | Decreased 1 | N/A | 13% | 14% | N/A | — | — |
| | category from | | | | | | |
| | baseline | | | | | | |

Results:

At BL, more patients on CVC than EFV had APRI≧0.5 and FIB-4≧1.45; proportion of CVC patients above these thresholds decreased at Weeks 24 and 48 (Table). Significant correlations were observed at Week 24 between changes in APRI score and MCP-1 levels (p=0.014), and between FIB-4 score and sCD14 levels (p=0.011), and at Week 48, between changes in APRI (p=0.028) and FIB-4 scores (p=0.007) and sCD14 levels.

Conclusions:

In this population with no apparent liver disease, CVC treatment was associated with improvements in APRI and FIB-4 scores, and correlations were observed between changes in APRI and FIB-4 scores and sCD14 levels at Week 48. Proven CCR2/CCR5 antagonism, antifibrotic effects in animal models and extensive clinical safety data all support clinical studies of CVC in liver fibrosis.

### Example 13: Cenicriviroc Achieves High CCR5 Receptor Occupancy at Low Nanomolar Concentrations

Background: Cenicriviroc (CVC) is a novel, once-daily, potent, CCR5 and CCR2 antagonist that has completed Phase 2b evaluation for the treatment of HIV-1 infection in treatment-naïve adults (NCT01338883). The aims of this study were to evaluate in vitro receptor occupancy and biology after treatment with CVC, BMS-22 (TOCRIS, a CCR2 antagonist) and an approved CCR5 antagonist, Maraviroc (MVC).

Methodology: PBMCs from 5 HIV+ and 5 HIV− subjects were incubated with CVC, BMS-22 or MVC, followed by either no treatment or treatment with a RANTES (CCR5 ligand) or MCP-1 (CCR2 ligand). The capacity of each drug to inhibit CCR5 or CCR2 internalization was evaluated. Cell-surface expression of CCR5 and CCR2 was assessed by flow cytometry, and fluorescence values were converted into molecules of equivalent soluble fluorescence (MESF).

Results:

Both CVC and MVC, in the absence of RANTES, increased cell-surface expression of CCR5. This effect was seen to a much greater degree in HIV-negative subjects (CD4+ and CD8+ T cells). CVC prevented RANTES-induced CCR5 internalization at lower effective concentrations than MVC. The effective concentration at which saturation of CCR5 was reached for CVC was 3.1 nM for CD4+ and 2.3 nM for CD8+ T cells (−91% and −90% receptor occupancy, respectively). MVC reached saturation at 12.5 nM for both CD4+ and CD8+ T cells, representing ˜86% and ˜87% receptor occupancy, respectively. CVC and MVC achieved high but incomplete saturation of CCR5, an effect that may be amplified by the observation of increased CCR5 expression with both agents in the absence of RANTES. In the absence of MCP-1, CVC induced CCR2 internalization and decreased cell-surface expression on monocytes. BMS-22 slightly increased CCR2 cell-surface expression. CVC prevented MCP-1-induced CCR2 internalization at lower concentrations than BMS-22. Saturation of monocyte CCR2 was reached at 6 nM of CVC, representing ˜98% CCR2 occupancy. To reach >80% receptor occupancy, an average of 18 nM of BMS-22 was required, compared to 1.8 nM of CVC.

Conclusions:

CVC more readily prevented RANTES-induced CCR5 internalization (at lower concentration) than MVC in vitro, indicating CVC more be more effective at preventing cellular activation by RANTES than MVC in vivo. Baseline CCR5 expression levels in treated subjects may be a determinant of CCR5 antagonist activity in vivo. CVC achieved ˜98% receptor occupancy of CCR2 on monocytes at low nanomolar concentrations in vitro, and reduced CCR2 expression on monocytes in the absence of MCP-1. High saturation of CCR2 by CVC paired with reduced expression may explain the potent CCR2 blockade observed with CVC in the clinic. CVC has potent immunomodulatory activities in vitro, and may be an important combined immunotherapeutic and anti-retroviral in chronic HIV infection.

### Example 14: CVC Blocks HIV Entry but does not Lead to Redistribution of HIV into Extracellular Space Like MVC

Background:

Cenicriviroc (CVC) is a novel, once-daily, dual CCR5/CCR2 co-receptor antagonist that has completed Phase 2b development. In Study 202 (NCT01338883), CVC demonstrated favorable safety and similar efficacy compared with the NNRTI efavirenz (EFV), when given in combination with emtricitabine/tenofovir (FTC/TDF). The CCR5 antagonist maraviroc (MVC) has been shown to sustain viral load levels in vitro by preventing HIV entry and repelling cell-free virions back into extracellular space. An ex vivo sub-analysis of Study 202 was conducted to establish if CVC prevents HIV entry, measured by intracellular HIV DNA declines, in subjects with virologic success at Week 24 (FDA Snapshot). In addition, in vitro assays were undertaken to determine and compare the extent of any cell-free virion redistribution that CVC or MVC may cause.

Methodology:

Ex vivo analysis: intracellular DNA was extracted from frozen PBMCs from 30 subjects with virologic success at Week 24 (10, 13 and 7 subjects on CVC 100 mg, CVC 200 mg and EFV 600 mg, respectively). Early (strong-stop) and late (full-length) reverse transcript levels were measured by qPCR. In vitro analysis: PM-1 cells were infected with CCR5-tropic HIV-1 BaL in the presence or absence of inhibitory concentrations of CVC (20 nM), MVC (50 nM) or controls. P24 and viral load levels were measured by ELISA and qRT-PCR after 4 hours.

Results:

Ex vivo analysis showed that full-length HIV DNA declines were similar across all groups (CVC 100 mg, CVC 200 mg and EFV 600 mg) at Week 24. Strong-stop HIV DNA declines (a marker of HIV entry) were more pronounced for both CVC arms (CVC 100 mg, 51% decline; CVC 200 mg, 37% decline) compared to the EFV arm (no decline) at Week 24. In vitro experiments revealed that cells treated with CVC exhibited considerably lower levels of supernatant P24 at 4 hours compared to baseline (0 hrs: 506 ng/ml; 4 hrs: 192 ng/ml). In contrast, supernatant P24 levels remained constant for MVC-treated cells after 4 hours (0 hrs: 506 ng/ml; 4 hrs: 520 ng/ml). Viral load levels for CVC-treated cells remained stable after 4 hours (0 hrs: 1.19×10¹⁰ copies/ml; 4 hrs: 1.26×10¹⁰ copies/ml). MVC-treated cells exhibited a slight increase in viral load after 4 hours (0 hrs: 1.19×10¹⁰ copies/ml; 4 hrs: 1.67×10¹⁰ copies/ml).

Conclusions:

Ex vivo analysis conducted in a subset of Study 202 subjects with virologic success at Week 24 confirmed that CVC treatment blocks HIV entry (strong-stop HIV DNA declines), while in vitro analysis showed that CVC-treated cells do not repel virus back into the extracellular space, as seen with MVC. Experiments are underway to determine whether or not direct interactions between CVC and HIV at the binding site may explain these unanticipated findings.

### Example 15: Receptor-Binding Properties of CVC and Metabolites

In addition, CVC has the unique property in vitro of being a CCR2 antagonist with 50% inhibitory concentrations (IC50) of 5.9 nmol/L. CVC dose-dependently inhibited the binding of RANTES, MIP-1α, and MIP-1β to CCR5-expressing Chinese hamster ovary (CHO) cells with an IC50 of 3.1, 2.3, and 2.3 nmol/L, respectively. CVC achieved ≧90% receptor occupancy for CCR5 at concentrations of 3.1 nM for CD4+ and 2.3 nM for CD8+ T-cells ex vivo in humans [4]. CVC inhibited the binding of MCP-1 to CCR2b with an IC50 of 5.9 nmol/L. CVC achieved ˜98% receptor occupancy for CCR2 on monocytes at 6 nM ex vivo in humans and reduced CCR2 expression on monocytes in the absence of MCP-1. CVC only weakly inhibited ligand binding to CCR3 and CCR4. CVC did not inhibit ligand binding to CCR1 or CCR7. CVC blocked RANTES-induced Ca2+ mobilization.

Two metabolites of CVC (M-I and M-II) were detected in animal studies (see Example 15); M-II was a major metabolite in monkeys and dogs, M-I was a minor metabolite in all species. M-I inhibited the binding of RANTES to CCR5-expressing cells with an IC50 of 6.5 nmol/L, which is approximately 2-fold the IC50 of CVC. M-II had no effect on binding of RANTES.

### Clinical Trials

### Example 16: Short-Term Efficacy Data in HIV-1 Infected Adult Subjects

### Methods

In the Phase 2a Study 201, antiviral activity of CVC was measured by changes in plasma HIV-1 RNA levels over 10 days of monotherapy, CD4⁺ cell counts, viral co-receptor shifts, and drug resistance testing.

Exploratory efficacy endpoints included changes in the inflammatory markers MCP-1, hs-CRP, and IL-6.

PK and PD were assessed by measurements of plasma drug concentrations up to 120 hours after the Day 1 and Day 10 CVC doses and trough plasma levels on Days 8 and 9.

### Design

Study 201 was a double-blind, randomized, placebo-controlled, dose-escalating Phase 2a study evaluating the antiviral activity, PK, safety, and tolerability of monotherapy of CVC for 10 days in subjects with CCR5-tropic HIV-1 infection. Subjects were antiretroviral treatment experienced, CCR5 antagonist-naïve, with HIV-1 RNA levels of at least 5000 copies/mL and CD4+ cell counts of at least 250 cells/mm³. Subjects with HIV-2, hepatitis B virus, or hepatitis C virus co-infection or dual/mixed-tropic HIV-1 were ineligible. Other than their HIV-1 infection, subjects were generally healthy. Groups of 10 subjects were sequentially enrolled in a ratio of 4:1 subjects per cohort to receive CVC (25, 50, 75, 100, or 150 mg) or matching placebo. All subjects received once-daily doses of CVC or placebo, with a meal, for 10 days and were followed to Day 40.

In this study, all dose groups received the original formulation (DP3_25), except the 100 mg dose group which received an alternative formulation (DP4_100). The subjects who received the 100 mg dose had significantly different PK results, and the formulation is no longer under development. These subjects were excluded from the efficacy, PK, and PK/PD data, but included in the baseline and safety data. In this study, safety and tolerability were assessed by monitoring AEs, vital signs, ECGs, clinical laboratory values from blood and urine, and physical examinations. Data from all subjects on placebo were pooled for the analyses.

### Adverse Events

CVC was generally well tolerated at doses between 25 mg and 150 mg with no SAEs, deaths, or other significant AEs, and there were no discontinuations because of an AE.

The most common (≧10%) treatment-emergent AEs were nausea (18.5%), diarrhea (16.7%), headache (14.8%), and fatigue (11.0%). Among subjects on CVC presenting with a AE (n=30), most experienced mild (80%) or moderate (17%) AEs. The only severe AE (abscess) was considered by the investigator as unrelated to study drug.

There were no indications of a dose relationship; however, subjects who received 150 mg of CVC (i.e., the highest dose studied) had more AEs compared to the subject in the other dose groups, although the severity of AEs was comparable across all dose groups. No AEs requiring treatment were considered probably related to study drug.

### Efficacy Results

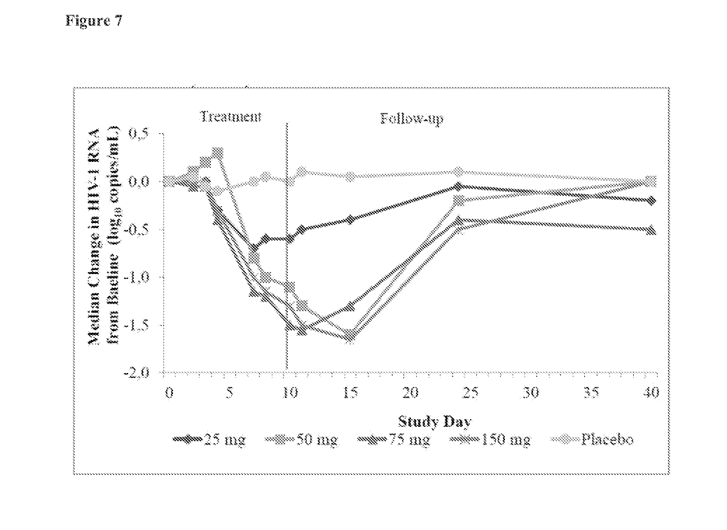

CVC showed a potent effect on HIV-1 RNA levels that persisted after completion of 10-day treatment. The mean changes in HIV-1 RNA levels are shown in FIG. 7. The mean HIV-1 RNA reductions from Baseline achieved statistical significance by Day 4 for all doses, and by Day 7 were p<0.002 for the 25 mg QD dose and p<0.001 the 50, 75, and 150 mg QD doses. This level of significance persisted through Day 15 for the 50 to 150 mg QD dose groups. HIV-1 RNA reductions were still significant at Day 24 in 150 mg dose group (p=0.03).

Median changes in HIV-1 RNA levels over time for all dose levels of CVC are summarized with descriptive statistics in Table 10. CVC exhibited potent antiviral activity, with the greatest median nadir values observed at the 75 and 150 mg QD dose levels. Over the entire study, 1 subject in the 25 mg dose group, 2 in the 50 mg dose group, and 3 in the 150 mg dose group achieved HIV-1 RNA levels <400 copies/mL. In addition, 2 subjects in the 150 mg dose group achieved HIV-1 RNA levels of <50 copies/mL in this 10-day monotherapy study. Mean and median changes in CD4+ cell counts were variable. Statistically significant increases from Baseline compared to placebo were only observed with the CVC 50 mg dose group on Day 7 (p=0.036) and Day 10 (p=0.020).

TABLE 10
Summary of HIV-1 RNA Levels by Cohort - Study 201

| | | CVC | CVC | CVC | CVC |
|---|---|---|---|---|---|
| Parameter | Placebo | 25 mg | 50 mg | 75 mg | 150 mg |
| Baseline | n = 10 | n = 9 | n = 7 | n = 8 | n = 8 |
| LS mean | 4.27 (0.17) | 4.46 (0.18) | 4.33 (0.21) | 4.65 (0.19) | 4.10 (0.19) |
| (SEM) | | | | | |
| Range | 3.2-5.1 | 3.1-6.0 | 3.9-4.7 | 4.3-5.3 | 3.6-4.9 |
| Day 11 | n = 10 | n = 9 | n = 7 | n = 8 | n = 8 |
| LS mean | −0.02 (0.14) | −0.64 (0.14) | −1.13 (0.16) | −1.46 (0.15) | −1.42 (0.16) |
| (SEM) | | | | | |
| change from | | | | | |
| Baseline | | | | | |
| p-value^{a} | N/A | 0.002 | <0.001 | <0.001 | <0.001 |
| Median | 0.1 | −0.5 | −1.3 | −1.6 | −1.5 |
| change from | | | | | |
| Baseline | | | | | |
| Nadir values | n = 10 | n = 9 | n = 7 | n = 8 | n = 8 |
| Median | −0.3 | −0.7 | −1.6 | −1.8 | −1.7 |
| change from | | | | | |
| Baseline | | | | | |
| Median | 5.5 | 10.0 | 11.0 | 10.0 | 11.0 |
| days to | | | | | |
| nadir | | | | | |

Values are expressed as log10 copies/mL.
Abbreviations: LS, least squares; SEM, standard error of the mean.
b P-values were one-sided based on comparison of the dose of CVC with placebo without multiple comparison adjustments. ANOVA was used with treatment as fixed factor at Baseline; analysis of covariance (ANCOVA) was used with treatment as fixed factor and baseline as covariate for change from Baseline.

As previously described, CVC has a dual activity as a CCR5 and CCR2 antagonist. Exploratory assessment of changes in MCP-1 (the ligand of CCR2, also known as CCL2), hs-CRP, and IL-6 were performed and significant dose-dependent increases in MCP-1 were observed (see Table 11). On Day 10, least square mean MCP-1 levels were 56.3, 94.2, 34.4, and 334.3 pg/mL higher than at Baseline in the 25, 50, 75, and 150 mg dose groups, respectively, compared to a slight decline in the placebo group. At the 50 and 150 mg doses, these results were statistically significant (p=0.024 and p<0.001, respectively). These results demonstrate the potent antagonistic CCR2 activity of CVC. CVC had no effect on hs-CRP or IL-6 levels overall in this 10-day study.

TABLE 11
Summary of MCP-1 Levels by Cohort - Study 201

| | | CVC | CVC | CVC | CVC |
|---|---|---|---|---|---|
| Parameter | Placebo | 25 mg | 50 mg | 75 mg | 150 mg |
| Baseline | n = 10 | n = 9 | n = 7 | n = 7 | n = 8 |
| Mean | 22.4 | 20.0 | 12.6 | 26.6 | 31.6 |
| Median | 18.5 | 16.0 | 6.0 | 8.0 | 19.5 |
| Range | 6-50 | 7-44 | 5-37 | 5-92 | 8-82 |
| Day 10, | n = 10 | n = 9 | n = 7 | n = 8 | n = 8 |
| pg/mL | | | | | |
| Mean | 21.0 | 75.3 | 101.3 | 59.1 | 372.0 |
| Median | 12.5 | 39.0 | 65.0 | 43.5 | 368.0 |
| Range | 5-52 | 10-287 | 21-266 | 20-128 | 79-605 |
| Change from | n = 10 | n = 9 | n = 7 | n = 7 | n = 8 |
| Baseline to | | | | | |
| Day 10 | | | | | |
| LS mean | −1.9 | +56.3 | +94.2 | +34.4 | +334.3 |
| P value^{a} | — | 0.095 | 0.024 | 0.222 | <0.001 |
| Median | 0.0 | +25.0 | +56.0 | +36.0 | +322.0 |

Abbreviation: LS, least squares
^{a}P-values were one-sided and based on comparison of each dose of CVC with placebo without multiple comparisons adjustment.

### Resistance Data

In Study 201, drug resistance testing was performed at Baseline, Day 7, and Day 40 (or at the “Early Termination” visit, if applicable). All subjects with evaluable samples remained fully susceptible to CVC.

### Viral Tropism

All subjects in Study 201 were tested for viral tropism to exclude that their virus was CXCR4 tropic or dual/mixed. All subjects had CCR5-tropic virus at screening (based on the enhanced sensitivity profile assay). A total of 39 subjects on CVC had evaluable samples following treatment, and one of these subjects (in the CVC 150 mg dose group) was found to have dual/mixed-tropic virus on Day 10. Further testing (at another laboratory using a different assay) revealed that this subject had mainly CXCR4-tropic virus at Baseline, therefore, this subject should not have been enrolled in the study according to the inclusion criteria. This subject did not respond to CVC treatment; the largest decrease in HIV-1 RNA of this subject was 0.13 log₁₀ copies/mL below the baseline value.

### Pharmacokinetic/Pharmacodynamic Relationships

For all doses tested in Study 201, a more than dose proportional increase in exposure was observed for “Formulation F1”, which was used for all but the 100 mg dose cohort.

Drug response was characterized using the following maximum effect (Eₘₐₓ) model:

$$E = {E}_{0} + \frac{( {I}_{max} - {E}_{0} ) \cdot {C}^{γ}}{{IC}_{50}^{γ} + {C}^{γ}}$$

where E is effect, E0 is the baseline effect (fixed to 0), Iₘₐₓ is the maximum inhibition, C denotes the PK variable (AUC₀₋₂₄, Cₘₐₓ, or steady-state concentration [Cₛₛ]), IC₅₀ is the value of the PK variable which corresponds to 50% of the maximum inhibition and γ is the shape parameter which describes the degree of sigmoidicity.

The Emax of CVC in the PK/PD model was −1.43 log₁₀ copies/mL. Based on the Emax model, average Cₛₛ of CVC for the 25, 50, 75, and 150 mg doses were expected to result in 54.9%, 79.8%, 85.9%, and 95.9% of the maximum inhibitory effect of the drug. Thus, dose levels of 75 and 150 mg QD displayed potent antiviral activity, with PD effects greater than 80% of the Eₘₐₓ of CVC in HIV-1-infected subjects.

### Example 17: Long-Term Efficacy Data in HIV-1 Infected Adult Subjects

### Efficacy Results of Study 202

### Week 24 Primary Analysis

The primary efficacy endpoint was the percentage of subjects with plasma HIV-1 RNA <50 copies/mL at Week 24 (using the FDA Snapshot algorithm and the ITT population).

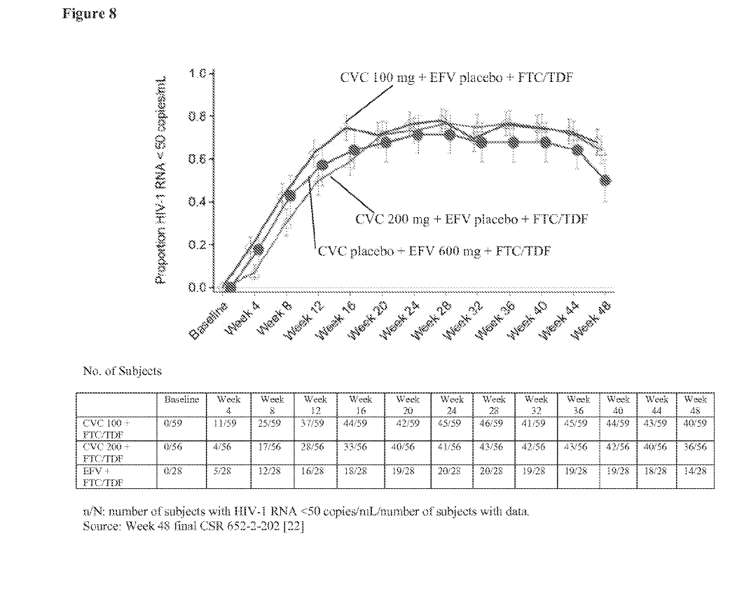

The percentage of subjects with virologic success at Week 24 was comparable among the 3 treatment arms: 76% with CVC 100 mg, 73% with CVC 200 mg, and 71% with EFV (all taken in combination with FTC/TDF) (see Table 11 and FIG. 8).

The percentage of subjects with virologic non-response was higher in the CVC arms (12% with CVC 100 mg and 14% with CVC 200 mg) than in the EFV arm (4%), and the percentage of subjects without virologic data at Week 24 was higher in the EFV arm (25%) than in the CVC arms (12% with CVC 100 mg and 13% with CVC 200 mg). The main reasons for discontinuation, and therefore for having virologic non-response or no virologic data, were adverse events in the EFV arm and meeting a mandatory protocol-defined withdrawal criteria for virologic failure in the CVC arms (prior to Protocol Amendment #6). Note that early discontinuations for reasons other than AEs and virologic failure also accounted for a large proportion of subjects with no virologic data at Week 24.

TABLE 11
Virologic Response (Plasma HIV-1 RNA <50 Copies/mL)
at Week 24 - Snapshot Algorithm - ITT - Study 202

| | CVC | | CVC | | | |
|---|---|---|---|---|---|---|
| | 100 mg | | 200 mg | | EFV | |
| n (%) | (N = 59) | | (N = 56) | | (N = 28) | |
| Virologic success | 45 | (76%) | 41 | (73%) | 20 | (71%) |
| (HIV-1 RNA <50 | | | | | | |
| copies/mL) | | | | | | |
| Treatment difference | 5% | | 4% | | — | |
| from EFV arm^{a} | | | | | | |
| (95% CI) | (−16%, 26%) | | (−17%, 25%) | | | |
| Virologic non-response^{b} | 7 | (12%) | 8 | (14%) | 1 | (4%) |
| HIV-1 RNA ≧50 | 3 | (5%)^{d} | 4 | (7%)^{e} | 1 | (4%)^{f} |
| but <400 copies/mL | | | | | | |
| HIV-1 RNA ≧400 | 4 | (7%) | 4 | (7%) | 0 | |
| copies/mL | | | | | | |
| No virologic data at | 7 | (12%) | 7 | (13%) | 7 | (25%) |
| Week 24, | | | | | | |
| reasons: | | | | | | |
| Discontinued study due | 0 | | 1 | (2%) | 5 | (18%) |
| to AE or death | | | | | | |
| Discontinued study for | 6 | (10%) | 6 | (11%) | 2 | (7%) |
| other reasons^{c} | | | | | | |

| | | | | |
|---|---|---|---|---|
| Missing data during | 1 | (2%)^{g} | 0 | 0 |
| Week 24 window, | | | | |
| but on study | | | | |

N = number of subjects; n = number of observations.
Note:
For this analysis, missing data were not imputed, but were considered as not having Virologic Success.
^{a}Treatment differences were estimated using stratum-adjusted Mantel-Haenszel proportions controlling for HIV-1 RNA at Baseline: 95% CIs were provided based on this method.
^{b}Includes subjects who changed therapy in a manner not permitted per protocol prior to Week 24, subjects who discontinued prior to Week 24 for lack or loss of efficacy, and subjects who had ≧50 copies/mL in the Week 24 window.
^{c}Other reasons included withdrawal of consent, loss to follow-up, moved, etc. Note that if subjects discontinued the study due to “Lack of Efficacy” or were using per protocol disallowed treatment during the study, the subjects were classified as having HIV-1 RNA >50 copies/mL (eg. virologic non-response).
^{d}Subjects 06006, 16004, and 52001.
^{e}Subjects 11003, 16030, 35011, and 56003.
^{f}Subject 66008
^{g}This subject (16014) did not have data in the Week 24 window. but had HIV-1 RNA <50 copies/mL at Weeks 12, 16 and 20, then had 102 copies/mL at Week 28 and <50 copies/mL from Week 32 onwards until Week 48 and FU.
Source: Week 48 final CSR 652-2-202 [22].

The proportion of subjects with virologic success (HIV-1 RNA <50 copies/mL at Week 24) was lower in the subgroup with high baseline viral load (≧100,000 copies/mL) in both CVC arms compared to the subgroup with low baseline viral load (≦100,000 copies/mL). However, although the numbers are small, the proportion of subjects with virologic non-response was similar across all treatment arms in the subgroup of subjects with high baseline viral load (≧100,000 copies/mL) (20% with CVC 100 mg, 29% with CVC 200 mg, and 25% EFV). In this subgroup with high baseline viral load, discontinuations for other reasons were only observed in both CVC arms (2 subjects in the CVC 100 mg arm and 3 subjects in the CVC 200 mg arm).

Virologic success appeared to be lower in subjects who were Black or African American compared to subjects who were not Black or African American, but this was most likely due to the relatively high rate of discontinuations in Black or African American subjects. No trends were observed for any other subgroups.

The results of the primary efficacy endpoint were confirmed by those of the secondary efficacy endpoints.

Week 48 Final Analysis

A greater proportion of CVC-than EFV-treated subjects completed the study, 42 (71%), 41 (73%), and 17 (61%) for the CVC 100 mg, CVC 200 mg, and EFV treatment arm, respectively. At Week 48, treatment with CVC was associated with higher rates of virologic success (HIV-1 RNA <50 copies/mL, FDA Snapshot analysis) but also with higher rates of virologic non-response compared to treatment with EFV. The percentages of subjects with virologic success (HIV-1 RNA <50 copies/mL) at Week 48 were: 68% with CVC 100 mg, 64% with CVC 200 mg, and 50% with EFV (see Table 12 and FIG. 8).

As observed at Week 24, at Week 48 the percentage of subjects with virologic non-response was higher in the CVC arms (15% with CVC 100 mg and 20% with CVC 200 mg) than in the EFV arm (11%), and the percentage of subjects without virologic data at Week 48 was higher in the EFV arm (39%) than in the CVC arms (17% with CVC 100 mg and 16% with CVC 200 mg). The main reasons for discontinuation in this analysis were the same as those in the Week 24 analysis.

As also seen in the Week 24 analysis, in the limited subgroup of subjects with high baseline viral load (≧100,000 copies/mL), the proportion of subjects with virologic non-response at Week 48 was similar across all treatment arms (20% with CVC 100 mg, 21% with CVC 200 mg, and 25% with EFV). In this small subgroup, a significant proportion of subjects had premature discontinuations for other reasons than virologic nonresponse or due to AEs (in both CVC arms) or had missing data (CVC and EFV arms), which limits the interpretability of these findings.

As observed in the Week 24 analysis, virologic success appeared to be lower in subjects who were Black or African American compared to subjects who were not Black or African American, but this was most likely due to the relatively high rate of discontinuations in Black or African American subjects. In fact, the proportion of CVC-treated Black or African American subjects with Week 48 non-response (8-15%) was comparable to that of the overall population (15-20%). In addition, although some differences were observed for gender, the proportion of female subjects enrolled in this study was too small to draw any conclusion. No trends were observed for any other subgroups.

TABLE 12
Virologic Response (Plasma HIV-1 RNA <50 Copies/mL)
at Week 48 Snapshot Algorithm - ITT - Study 202

| | CVC | | CVC | | | |
|---|---|---|---|---|---|---|
| | 100 mg | | 200 mg | | EFV | |
| n (%) | (N = 59) | | (N = 56) | | (N = 28) | |
| Virologic success | 40 | (68%) | 36 | (64%) | 14 | (50%) |
| (HIV-1 RNA <50 | | | | | | |
| copies/mL) | | | | | | |
| Treatment difference | 18% | | 16% | | — | |
| from EFV arm^{a} | | | | | | |
| (95% CI) | (−5%, 41%) | | (−7%, 39%) | | | |
| Virologic non-response^{b} | 9 | (15%) | 11 | (20%) | 3 | (11%) |
| No virologic data at | 10 | (17%) | 9 | (16%) | 11 | (39%) |
| Week 48, | | | | | | |
| reasons: | | | | | | |
| Discontinued study due | 0 | | 1 | (2%) | 6 | (21%) |
| to AE or death | | | | | | |
| Discontinued study for | 8 | (14%) | 7 | (13%) | 3 | (11%) |
| other reasons^{c} | | | | | | |
| Missing data during | 2 | (3%) | 1 | (2%) | 2 | (7%) |
| Week 48 window. | | | | | | |
| but on study | | | | | | |

N = number of subjects; n = number of observations.
Note:
For this analysis, missing data were not imputed, but were considered as not having Virologic Success.
^{a}Treatment differences were estimated using stratum-adjusted Mantel-Haenszel proportions controlling for HIV-1 RNA at Baseline: 95% CIs were provided based on this method.
^{b}Includes subjects who changed therapy in a manner not permitted per protocol prior to Week 48, subjects who discontinued prior to Week 48 for lack or loss of efficacy, and subjects who had ≧50 copies/mL in the Week 48 window.
^{c}Other reasons included withdrawal of consent, loss to follow-up, moved, etc.
Note that if subject discontinued the study due to “Lack of Efficacy” or were using per protocol disallowed treatment dureing the study, the subjects were classified as having HIV-1 RNA >50 copies/mL (eg, virologic non-response).
Source: Week 48 final CSR 652-2-202 [22].

### Example 18: HIV-2 In Vitro Susceptibility to CCR5 Inhibitors/Antagonists

Background:

HIV-2 is naturally resistant to non-nucleoside reverse transcriptase inhibitors, fusion inhibitor, and to some protease inhibitors. Maraviroc (MVC), the only approved CCR5 antagonist, is effective against CCR5-tropic (R5) HIV-1 and HIV-2 infections. Cenicriviroc (CVC), a novel, once-daily, dual CCR5 and CCR2 antagonist, has completed Phase 2b development for treatment of HIV-1 infection (NCT01338883). Until now, HIV-2 susceptibility to CVC had not been evaluated. In this study we evaluated HIV-2 susceptibility to cenicriviroc in a PBMC culture model.

The susceptibility of HIV-2 to the CCR5 inhibitors/antagonists Maraviroc (MVC) and Cenicriviroc (CVC) were tested. To test susceptibility of HIV-2 to CVC, we analyzed six HIV-2 clinical isolates issued from six patients by PBMC culture. All patients were infected with HIV-2 group A. Tropism was determined phenotypically using Ghost(3) cell lines expressing CD4 receptor and one HIV coreceptor, CCR5 or CXCR4. Both Ghost(3) cell lines carries a humanized green fluorescent protein (GFP) gene, under the control of a HIV-2 LTR. Infected cells can be detected by cytometric analysis (Visseaux et al. (2012) J. Infect. Dis.). Ghost(3) cells were provided by the NIH AIDS Reagent program. All viral strains were previously tested for maraviroc susceptibility (Visseaux et al. (2012) Antimicrob. Agents. Chemother).

Phenotypic susceptibility of HIV-2 clinical isolates to cenicriviroc was determined using modified version of the ANRS PBMC method previously used for HIV-2 susceptibility to maraviroc (Visseaux et al. (2012) Antimicrob. Agents. Chemother).

Briefly, cell-free HIV-2 positive supernatant was serially diluted (1, 10⁻¹, and 10⁻²) and incubated with PBMC for viral titration. Viruses were tested at a 100 TCID₅₀ titration. Cells were pre-incubated with cenicriviroc or maraviroc at an appropriate concentration 1 hour before viral infection. Six serial dilutions of the antiretroviral drug were tested in quadruplicate. On day 4, viral replication was assessed by a specific viral load assay (Generic HIV-2 viral load, Biocentric, France). Drug concentrations inhibiting 50% of the replication (EC50) and maximum percentage inhibition (MPI) were measured.

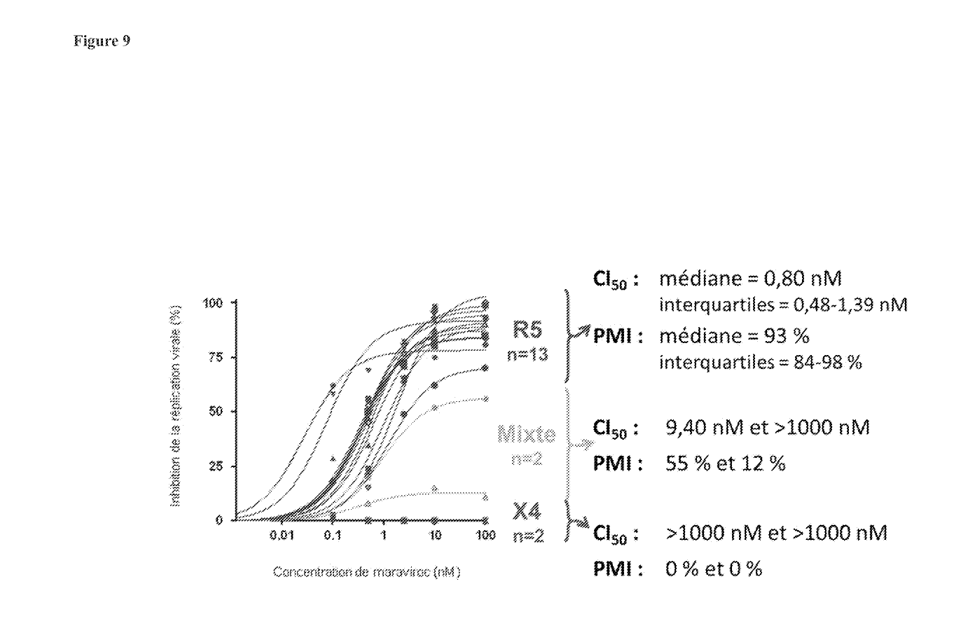

FIG. 9 shows the inhibition of HIV-2 viral replication after exposure to maraviroc. For the thirteen R5 clinical isolates, the median EC₅₀ is 0.80 nM, with the interquartiles of 0.48-1.39 nM; the median MPI is 93%, with the interquartiles of 84-98%. For the two mixed R5/X4 clinical isolates, the median EC₅₀ is 9.40 nM and greater 1000 nM, and the median MPI is 55% and 12%. For the X4 clinical isolate, the median EC₅₀ is greater than 1000 nM, and the median MPI is 0%.

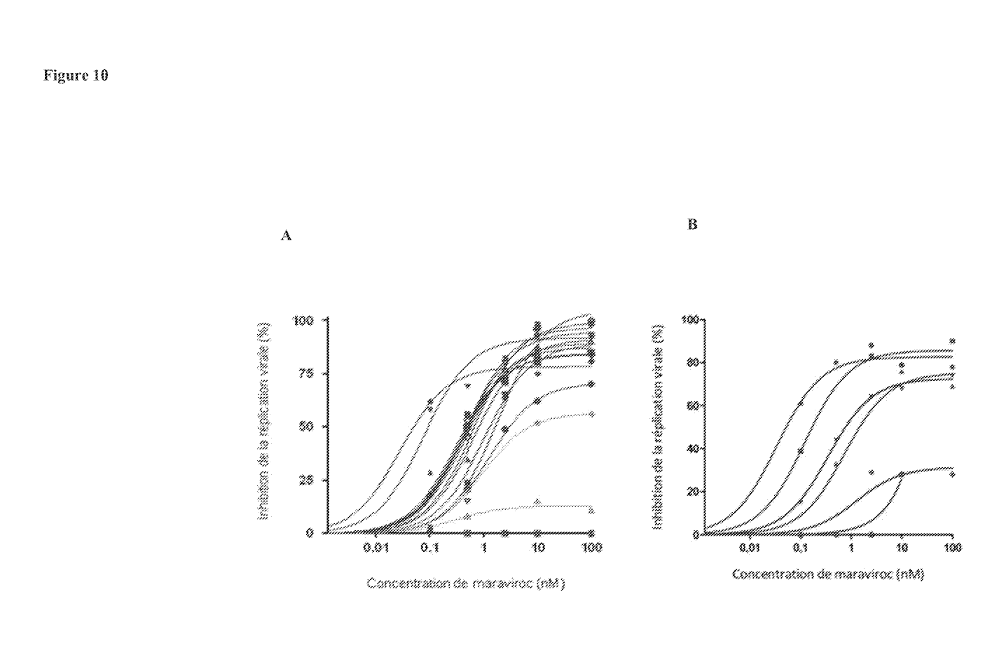

Table 13 shows a comparison of the median EC₅₀ and MPIs for HIV-2 and HIV-1 we observed with that for HIV-1 observed by Dorr et al. (2005). FIGS. 10 A and B shows the percent viral inhibition for HIV-2 to MVC (Panel A) and HIV-1 (Panel B).

TABLE 13

| | Observed Results | | Dorr et al. (2005) |
|---|---|---|---|
| | HIV-2 (n = 13) | HIV-1 (n = 4) | HIV-1 (n = 43 |
| Median IC₅₀ | 0.8 nM | 2.4 nM | 0.4 nM |
| (IQR) | (0.5-1.4) | (0.8-4.2) | (0.3-1.0) |
| Median MPI | 93% | 74% | — |
| (IQR) | (84-98) | (73-81) | |

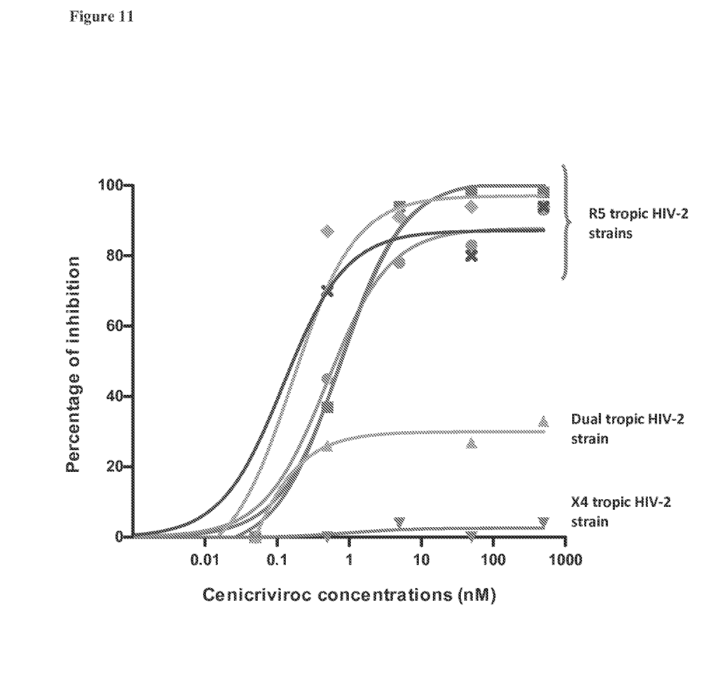

Table 14 shows the cenicriviroc and maraviroc half maximal effective concentration (EC50) and maximum percentage inhibition (MPI) among tested HIV-2 primary clinical isolates. FIG. 11 shows the percentage of HIV-2 viral inhibition by CVC.

TABLE 14

| | | Tropism phenotypic assay [4] | | | Maraviroc susceptibility | | Cenicriviroc susceptibility | |
|---|---|---|---|---|---|---|---|---|
| | Viral | RTCN * | | Viral | EC50 | MPI | EC50 | MPI |
| Virus | group | CCR5 | CXCR4 | tropism | (nM) | (%) | (nM) | (%) |
| 10-046 | A | 99 | — | R5 | 1.13 | 93 | 0.03 | 94 |
| 10-051 | A | 373 | — | R5 | 0.48 | 82 | 0.45 | 93 |
| 10-056 | A | 648 | 32 | R5 | 0.58 | 90 | 0.33 | 94 |
| 10-074 | A | 769 | — | R5 | 0.68 | 100 | 0.98 | 98 |
| 10-069 | A | 779 | 300 | Dual | >1000 | 12 | >1000 | 33 |
| 10-055 | A | 19 | 206 | X4 | >1000 | 0 | >1000 | 4 |

* RTCN: Ratio to Cell Negative

For the four R5 HIV-2 clinical isolates tested, the EC₅₀ for CVC were 0.03, 0.45, 0.33 and 0.98 nM with MPI at 94, 93, 94 and 98%. These values are similar to those observed with MVC with EC₅₀ and MPI at 1.13, 0.48, 0.58 and 0.68 nM and 93, 82, 90 and 100%, respectively. The dual and X4 tropic HIV-2 strains were resistant to CVC with EC50 and MPI of >1000 nM, 33% and >1000 nM, 4%, respectively.

We demonstrated for the first time that cenicriviroc is active in vitro on HIV-2 R5 tropic strains with similar EC50 and MPI to those observed with maraviroc. Once-daily CVC treatment may expand the limited therapeutic arsenal for HIV-2-infected patients. Clinical studies are warranted.

The detailed description herein describes various aspects and embodiments of the invention, however, unless otherwise specified, none of those are intended to be limiting. Indeed, a person of skill in the art, having read this disclosure, will envision variations, alterations, and adjustments that can be made without departing from the scope and spirit of the invention, all of which should be considered to be part of the invention unless otherwise specified. Applicants thus envision that the invention described herein will be limited only by the appended claims.

### REFERENCES

1. Saiman Y, Friedman S L. The role of chemokines in acute liver injury. 2012; 3:213.
2. Zimmermann H W, Tacke F. Modification of chemokine pathways and immune cell infiltration as a novel therapeutic approach in liver inflammation and fibrosis. Inflamm Allergy Drug Targets. 2011; 10:509-536.
3. Seki E, De Minicis S, Gwak G Y, Kluwe J, Inokuchi S, Bursill C A, Llovet J M, Brenner D A, Schwabe R F. CCR1 and CCR5 promote hepatic fibrosis in mice. J Clin Invest. 2009; 119:1858-1870.
4. Seki E, de Minicis S, Inokuchi S, Taura K, Miyai K, van Rooijen N, Schwabe R F, Brenner D A. CCR2 promotes hepatic fibrosis in mice. Hepatology. 2009; 50:185-197.
5. Miura K, Yang L, van Rooijen N, Ohnishi H, Seki E. Hepatic recruitment of macrophages promotes nonalcoholic steatohepatitis through CCR2. Am J Physiol Gastrointest Liver Physiol. 2012; 302:G1310-1331.
6. Ochoa-Callejero L, Perez-Martinez L, Rubio-Mediavilla S, Oteo J A, Martinez A, Blanco J R. Maraviroc, a CCR5 antagonist, prevents development of hepatocellular carcinoma in a mouse model. PLoS One. 2013; 8:e53992.
7. Mitchell C, Couton D, Couty J P, Anson M, Crain A M, Bizet V, Rénia L, Pol S, Mallet V, Gilgenkrantz H. Dual role of CCR2 in the constitution and the resolution of liver fibrosis in mice. Am J Pathol. 2009; 174:1766-1775.
8. Xia Y, Entman M L, Wang Y. CCR2 regulates the uptake of bone marrow-derived fibroblasts in renal fibrosis. PLoS ONE 2013; 8(10): e77493. doi:10.1371/journal.pone.0077493
9. Karlmark K R, Wasmuth H E, Trautwein C, Tacke F. Chemokine-directed immune cell infiltration in acute and chronic liver disease. Expert Review Gastroenterology Hepatology. 2008; 2: 233-242
10. Vielhauer V, Anders H-J, Mack M, Cihak J, Strutz F, Stangassinger M, Luckow B, Gröne H-J, Schlöndorff D. Obstructive nephropathy in the mouse: Progressive fibrosis correlates with tubulointerstitial chemokine expression and accumulation of CC chemokine receptor 2- and 5-positive leukocytes. J Am Soc Nephrol 2001; 12:1173-1187.
11. Segerer S, Mack M, Regele H, Kerjaschki D, Schlöndorff D. Expression of the C—C chemokine receptor 5 in human kidney disease. Kidney Int 1999; 56:52-64.
12. Wai C-T, Greenson J, Fontana R, Kalbfleisch J, Marrero J, Conjeevaram H, Lok A. A simple noninvasive index can predict both significant fibrosis and cirrhosis in patients with chronic hepatitis C. Hepatology 2003; 38:518-526.
13. Vallet-Pichard A, Mallet V, Nalpas B, Verkarre V, Nalpas A, Dhalluin-Venier V, Fontaine H, Pol S. FIB-4: an inexpensive and accurate marker of fibrosis in HCV infection. Comparison with liver biopsy and FibroTest. Hepatology 2007; 46:32-36.
14. Sandler N G, Wand H, Roque A, et al. Plasma levels of soluble CD14 independently predict mortality in HIV infection. JID 2011; 203:780-790.
15. Brenchley J, Price D A, Schacker T W, Asher T E, Silvestri G, Rao S, Kazzaz Z, Bornstein E, Lambotte O, Altmann D, Blazar B R, Rodriguez B, Teixeira-Johnson L, Landay A, Martin J N, Hecht F M, Picker L J, Lederman M M, Deeks S G, Douek D C. Microbial translocation is a cause of systemic immune activation in chronic HIV infection. Nature Medicine 2006; 12:1365-1371.
16. Vajro P, Paolella G, Fasano A. Microbiota and Gut-Liver Axis: Their Influences on Obesity and Obesity-Related Liver Disease JPGN 2013; 56: 461-468.
17. Roh Y S, Seki E. Toll-like receptors in alcoholic liver disease, non-alcoholic steatohepatitis and carcinogenesis Journal of Gastroenterology and Hepatology 2013; 28: 38-42.
18. Ilan Y. Leaky gut and the liver: A role for bacterial translocation in nonalcoholic steatohepatitis. World J Gastroenterol 2012 Jun. 7; 18: 2609-2618.
19. Petrasek J, Mandrekar P, Szabo G. Toll-Like Receptors in the Pathogenesis of Alcoholic Liver Disease. Gastroenterology Research and Practice 2010, Article ID 710381, 12 pages. doi:10.1155/2010/710381.
20. Sandler N, Koh C, Roque A, Eccleston J, Siegel R, Demino M, Kleiner D, Deeks S, Liang T-J, Heller T, Douek D. Host Response to Translocated Microbial Products Predicts Outcomes of Patients With HBV or HCV Infection. Gastroenterology 2011; 141:1220-1230. doi:10.1053/j.gastro.2011.06.063.
21. Wiest R, Lawson M, Geuking M. Pathological bacterial translocation in liver cirrhosis. J Hepatology 2014; 60(1):197-209.
22. Shanab A A, Scully P, Crosbie O, Buckley M, O'Mahony L, Shanahan F, Gazareen S, Murphy E, Quigley E M. Small intestinal bacterial overgrowth in nonalcoholic steatohepatitis: association with toll-like receptor 4 expression and plasma levels of interleukin 8. Dig Dis Sci 2011; 56: 1524-1534
23. Henao-Mejia J, Elinav E, Jin C, Hao L, Mehal W Z, Strowig T, Thaiss C A, Kau A L, Eisenbarth S C, Jurczak M J, Camporez J-P, Shulman G I, Gordon J I, Hoffman H M; Flavell R A. Inflammasome-mediated dysbiosis regulates progression of NAFLD and obesity. Nature 2012; 492: 179-185. doi:10.1038/nature10809.
24. Klibanov, Olga M.; Williams, Shannon H.; Iler, Cameron A (2010). “Cenicriviroc, an orally active CCR5 antagonist for the potential treatment of HIV infection”. Current Opinion in Investigational Drugs 11 (8): 940-950.
25. Kleiner D E. et al., Hepatology, Design and validation of a histological scoring system for nonalcoholic fatty liver disease; 2005; 41:1313-1321.
26. Fujii H et al. J. Atheroscler. Thromb. 2009; 16:893.
27. Rangwala F et al. J. Pathol. 2011; 224:401.
28. Seki et al. CCR1 and CCR5 promote hepatic fibrosis in mice. J. Clin. Invest. 2009; 119(7):1858-1870.
29. Xu et al. Liver fibrosis: mechanisms of immune-mediated liver injury. Cellular & Mol. Immunol.; 2012; 9:296-301.
30. Karlmark et al, Expert Rev. Gastroenterol. Hepatol. 2(2), 233-242 (2008).
31. Visseaux et al. Antimicrob. Agents Chemother., 56(1):137 (2012).
32. Damond et al. J. Clin, Microbiol. 43:4234-4236 (2005).
33. Whittle et al. AIDS. 8:1617-1620 (1994).
34. Reeves et al. J Virol. 73(9):7795-7804 (1999).
35. Liu R, Paxton W A, Choe S, Ceradini D, Martin S R, Horuk R, et al. Homozygous defect in HIV-1 coreceptor accounts for resistance of some multiply-exposed individuals to HIV-1 infection. Cell 1996; 86(3):367-77.
36. Samson M, Libert F, Doranz B J, Rucker J, Liesnard C, Farber C M, et al. Resistance to HIV-1 infection in Caucasian individuals bearing mutant alleles of the CCR-5 chemokine receptor gene. Nature 1996; 382(6593):722-5.
37. Visseaux B, Hurtado-Nedelec M, Charpentier C, et al., Molecular determinants of HIV-2 R5-X4 tropism in the V3 loop: development of a new genotypic tool, J. Infect. Dis., 2012, 205(1) p111-20.
38. Espirito-Santo M, Santos-Costa Q, Calado M, et al. Susceptibility of HIV Type 2 Primary Isolates to CCR5 and CXCR4 Monoclonal Antibodies, Ligands, and Small Molecule Inhibitors, AIDS Res. Hum. Retroviruses 2012; 28:478-485.
39. Borrego P, Calado R, Marcelino J M, et al. Baseline susceptibility of primary HIV-2 to entry inhibitors, Antivir. Ther. 2012; 17:565-570.

1. A method of treating HIV-2 infectious disease in a patient in need of such treatment, comprising administering to the patient a therapeutically effective amount of a compound of formula (I): 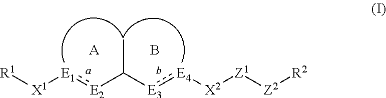
wherein
R¹ is a cyclic 5- to 6-membered ring which may be substituted;
X¹ is a bond;
rings A and B, together with the variables a, b, E₁, E₂, E₃, and E₄, form a benzoazocine ring system;
X² is a bivalent chain group whose straight chain moiety is constituted of 1 to 4 atoms;
Z¹ is a bond or a bivalent cyclic group;
Z² is a bond or a bivalent group; and
R² is
(1) an amino group which may be substituted and whose nitrogen atoms may be converted to quaternary ammonium or oxide,
(2) a nitrogen-containing heterocyclic group which may be substituted, may contain a sulfur or oxygen atom as a ring constituent atom, and whose nitrogen atom may be converted to quaternary ammonium or oxide,
(3) a group of the formula: 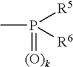
wherein k is 0 or 1; when k is 0, the phosphorus atom may form a phosphonium salt; each of R⁵ and R⁶ is a hydrocarbon group which may be substituted, a hydroxy group or an amino group which may be substituted; and R⁵ and R⁶ may form a ring with the adjacent phosphorus atom,
(4) an amidino group which may be substituted, or
(5) a guanidino group which may be substituted;
or a salt thereof, to a mammal in need thereof.

2. The method of claim 1, wherein the compound of formula (I) is administered orally.

3. The method of claim 1 or 2 comprising adding the compound to blood for transfusion or to blood derivatives in combination with one or more agents that purge latent HIV reservoirs.

4. The method of claim 3, wherein the compound is administered at the same time of or within 1 hour after transfusion or use of blood derivatives.

5. The method of claim 1, wherein the compound of formula (I) is (S)-(8)-[4-(2-Butoxyethoxy)phenyl]-1-isobutyl-N-(4-{[(1-propyl-1H-imidazol-5-yl)methyl]sulfinyl}phenyl)-1,2,3,4-tetrahydro-1-benzazocine-5-carboxamide or a salt thereof.

6. The method of claim 5, wherein the compound of formula (I) is (S)-(8)-[4-(2-Butoxyethoxy)phenyl]-1-isobutyl-N-(4-{[(1-propyl-1H-imidazol-5-yl)methyl]sulfinyl}phenyl)-1,2,3,4-tetrahydro-1-benzazocine-5-carboxamide monomethanesulfonoate.

7. A method of treating HIV-2 infectious disease in a patient in need of such treatment, comprising administering to the patient a therapeutically effective amount of a compound of formula (I): 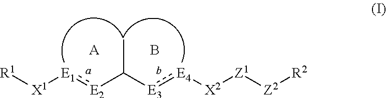
wherein
R¹ is a cyclic 5- to 6-membered ring which may be substituted;
X¹ is a bond;
rings A and B, together with the variables a, b, E₁, E₂, E₃, and E₄, form a benzoazocine ring system;
X² is a bivalent chain group whose straight chain moiety is constituted of 1 to 4 atoms;
Z¹ is a bond or a bivalent cyclic group;
Z² is a bond or a bivalent group; and
R² is
(1) an amino group which may be substituted and whose nitrogen atoms may be converted to quaternary ammonium or oxide,
(2) a nitrogen-containing heterocyclic group which may be substituted, may contain a sulfur or oxygen atom as a ring constituent atom, and whose nitrogen atom may be converted to quaternary ammonium or oxide,
(3) a group of the formula: 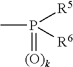
wherein k is 0 or 1; when k is 0, the phosphorus atom may form a phosphonium salt; each of R⁵ and R⁶ is a hydrocarbon group which may be substituted, a hydroxy group or an amino group which may be substituted; and R⁵ and R⁶ may form a ring with the adjacent phosphorus atom,
(4) an amidino group which may be substituted, or
(5) a guanidino group which may be substituted;
or a salt thereof, to a mammal in need thereof.

8. The method of claim 7, wherein the compound of formula (I) is administered orally.

9. The method of claim 7 or 8 comprising adding the compound to blood for transfusion or to blood derivatives in combination with one or more agents that purge latent HIV reservoirs.

10. The method of claim 9, wherein the compound is administered at the same time of or within 1 hour after transfusion or use of blood derivatives.

11. The method of claim 7, wherein the compound of formula (I) is (S)-(8)-[4-(2-Butoxyethoxy)phenyl]-1-isobutyl-N-(4-{[(1-propyl-1H-imidazol-5-yl)methyl]sulfinyl}phenyl)-1,2,3,4-tetrahydro-1-benzazocine-5-carboxamide or a salt thereof.

12. The method of claim 11, wherein the compound of formula (I) is (S)-(8)-[4-(2-Butoxyethoxy)phenyl]-1-isobutyl-N-(4-{[(1-propyl-1H-imidazol-5-yl)methyl]sulfinyl}phenyl)-1,2,3,4-tetrahydro-1-benzazocine-5-carboxamide monomethanesulfonoate.

13. A method of treating HIV-2 infectious disease in a patient in need of such treatment, comprising administering to the patient a therapeutically effective amount of a compound of formula (I): 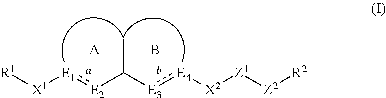
wherein
R¹ is a cyclic 5- to 6-membered ring which may be substituted;
X¹ is a bond;
rings A and B, together with the variables a, b, E₁, E₂, E₃, and E₄, form a benzoazocine ring system;
X² is a bivalent chain group whose straight chain moiety is constituted of 1 to 4 atoms;
Z¹ is a bond or a bivalent cyclic group;
Z² is a bond or a bivalent group; and
R² is
(1) an amino group which may be substituted and whose nitrogen atoms may be converted to quaternary ammonium or oxide,
(2) a nitrogen-containing heterocyclic group which may be substituted, may contain a sulfur or oxygen atom as a ring constituent atom, and whose nitrogen atom may be converted to quaternary ammonium or oxide,
(3) a group of the formula: 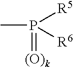
wherein k is 0 or 1; when k is 0, the phosphorus atom may form a phosphonium salt; each of R⁵ and R⁶ is a hydrocarbon group which may be substituted, a hydroxy group or an amino group which may be substituted; and R⁵ and R⁶ may form a ring with the adjacent phosphorus atom,
(4) an amidino group which may be substituted, or
(5) a guanidino group which may be substituted;
or a salt thereof, to a mammal in need thereof.

14. The method of claim 13, wherein the compound of formula (I) is administered orally.

15. The method of claim 13 or 14 comprising adding the compound to blood for transfusion or to blood derivatives in combination with one or more agents that purge latent HIV reservoirs.

16. The method of claim 15, wherein the compound is administered at the same time of or within 1 hour after transfusion or use of blood derivatives.

17. The method of claim 7, wherein the compound of formula (I) is (S)-(8)-[4-(2-Butoxyethoxy)phenyl]-1-isobutyl-N-(4-{[(1-propyl-1H-imidazol-5-yl)methyl]sulfinyl}phenyl)-1,2,3,4-tetrahydro-1-benzazocine-5-carboxamide or a salt thereof.

18. A method of inhibiting HIV-2 binding to a cell comprising administering an effective amount of a salt according to claim 14 to a subject in need thereof.

19. The method of claim 11 or 17, wherein the HIV-2 binding comprises binding of HIV-2 to a cell-surface receptor.

20. The method of claim 19, wherein the binding of HIV-2 to a cell-surface receptor is blocked and/or inhibited.

21. The method of claim 19, wherein the cell-surface receptor is CCR5.
